(19) United States
(12) Patent Application Publication
(10) Pub. No.: US 2015/0037428 A1
(43) Pub. Date: Feb. 5, 2015

(54) GEOMETRICALLY ENGINEERED PARTICLES AND METHODS FOR MODULATING MACROPHAGE OR IMMUNE RESPONSES

(71) Applicants: The University of North Carolina at Chapel Hill, Chapel Hill, NC (US); Liquidia Technologies, Inc., Durham, NC (US)

(72) Inventors: Joseph DeSimone, Chapel Hill, NC (US); Mary Elizabeth Napier, Chapel Hill, NC (US); Chris Luft, Chapel Hill, NC (US); Pete Mack, Chapel Hill, NC (US); Ben Maynor, Durham, NC (US); Tammy Shen, Chapel Hill, NC (US)

(21) Appl. No.: 14/361,138

(22) PCT Filed: Nov. 28, 2012
(86) PCT No.: PCT/US12/66790

§ 371 Date: May 28, 2014

Related U.S. Application Data
(60) Provisional application No. 61/564,626, filed on Nov. 29, 2011.

(51) Int. Cl. A61K 9/00; G01N 33/50; A61K 9/14

(52) U.S. Cl. CPC A61K 9/007; A61K 9/14; G01N 33/5047; G01N 2500/10
USPC 424/501; 424/280.1; 435/29

(57) ABSTRACT

Disclosed herein are geometrically engineered particles having varied shapes and sizes and surface charge which can incorporate drugs and/or other biomaterials for targeted delivery, such as pulmonary delivery. The size, shape, etc. of a particle can be designed and corresponding particles can be prepared that target or de-target immunological responses to the particles themselves, for example, the response of alveolar macrophages. Methods of modulating immune responses by utilizing the particles are also disclosed. The particles can be composed substantially of therapeutic, drug and polymer or can comprise polymers and proteins. The particles may also be composed of diagnostic agents and additional biomaterials to confer aerosolization and cellular uptake properties. The particles also may have a range of physical features such as fenestrations, angled arms, asymmetry and surface roughness, charge which alter the interactions with cells and tissues.

### GOVERNMENT SUPPORT

This invention was made with government support under Grants 1DP1OD006432 awarded by the National Institutes of Health. The government has certain rights in the invention.

### FIELD OF THE INVENTION

The subject matter herein is directed to geometrically engineered particles that can be tailored to target or de-target immune responses such as macrophage and mast cell responses. Methods of modulating immune responses utilizing the particles are also disclosed.

### BACKGROUND

Drug delivery technology has been exploited extensively for the purpose of delivering agents to desired targets for many years. Drug delivery technologies involve liposomes and nano or microparticles. Hydrophobic or hydrophilic compounds can be entrapped in the hydrophobic domain or encapsulated in the aqueous compartment, respectively. Liposomes can be constructed of natural constituents so that the liposome membrane is in principal identical to the lipid portion of natural cell membranes. It is considered that liposomes are quite compatible with the human body when used as drug delivery systems.

The cellular delivery of various therapeutic compounds, such as chemotherapeutic agents, is usually compromised by two limitations. First, the selectivity of a number of therapeutic agents is often low, resulting in high toxicity to normal tissues. Secondly, the trafficking of many compounds into living cells is highly restricted by the complex membrane systems of the cell. Specific transporters allow the selective entry of nutrients or regulatory molecules, while excluding most exogenous molecules such as nucleic acids and proteins.

Aerosolized medicaments are used to treat patients suffering from a variety of respiratory ailments. Medicaments can be delivered directly to the lungs by having the patient inhale the aerosol through a tube and/or mouthpiece coupled to the aerosol generator. By inhaling the aerosolized medicament, the patient can quickly receive a dose of medicament that is concentrated at the treatment site (e.g., the bronchial passages and lungs of the patient). Generally, this is a more effective and efficient method of treating respiratory ailments than first administering a medicament through the patient's circulatory system (e.g., intravenous injection). However, may problems still exist with the delivery of aerosolized medicaments.

### SUMMARY OF THE INVENTION

Disclosed herein are geometrically engineered particles having varied shapes and sizes and surface charge which can incorporate drugs and/or other biomaterials for targeted delivery, such as pulmonary delivery. The size, shape, etc. of a particle can be designed and corresponding particles can be prepared that target or de-target immunological responses to the particles themselves, for example, the response of alveolar macrophages. Methods of modulating immune responses by utilizing the particles are also disclosed. The particles can be composed substantially of therapeutic, drug and polymer or can comprise polymers and proteins. The particles may also be composed of diagnostic agents and additional biomaterials to confer aerosolization and cellular uptake properties. The particles also may have a range of physical features such as fenestrations, angled arms, asymmetry and surface roughness, charge which alter the interactions with cells and tissues.

### BRIEF DESCRIPTION OF THE FIGURES

FIG. 1A-G shows SEM images of certain representative shapes of particles described herein.

FIG. 2A-C are graphical data comparing alveolar macrophage uptake of different shapes using FACS analysis. The data show the internalization profile of PRINT particles in (A) MH-S (murine alveolar macrophages) and (B) RAW264.7 (murine leukaemic monocyte) cells. Doubling time of RAW cells is twice as fast as MH-S which may account for the dip in internalization % at 24 hours. Data show similar internalization trends in different cell lines.

FIG. 3 shows internalization of particles by MH-S cells plotted by volume at select time points.

FIG. 4A-C shows still image time lapse of cellular internalization. The particle first contacted by the cell on “ball” section is fully internalized while the particle contacted at the angled “stick” section is still attached to the outside of the cell membrane.

FIG. 5A-B shows micrographs of MH-S cells associating with shaped PEG particles depicting local particle angle effects on phagocytosis. (A) 9.54 μm L-dumbbells, (B, F) 6 μm donuts, (C, E) 11.68 μm pollen, (D) 10.24 μm helicopter.

FIG. 6A-F shows the local particle contact angle effects. Most observed particles in the process of being internalized had Ω<45°.

FIG. 7 depicts (A) Determination of particle internalization orientation using fluorescent microscopy (Orientation labels—S: stick, B: ball, I: fully internalized, U: Undetermined/Sideways); (B) Bar graph of lollipop particle internalization orientation at 0.5 hours in MH-S macrophages. Corresponding SEM images of particle orientation also shown.

FIG. 8A-B shows micrographs depicting the ability of macrophages to deform highly cross-linked PEG particles; (A) V-boomerangs being stretched into a more linear particle by two cells; (B) Helicopter shape drawn towards the cell membrane.

FIG. 9 depicts pulmonary relevance of the particles and methods described herein.

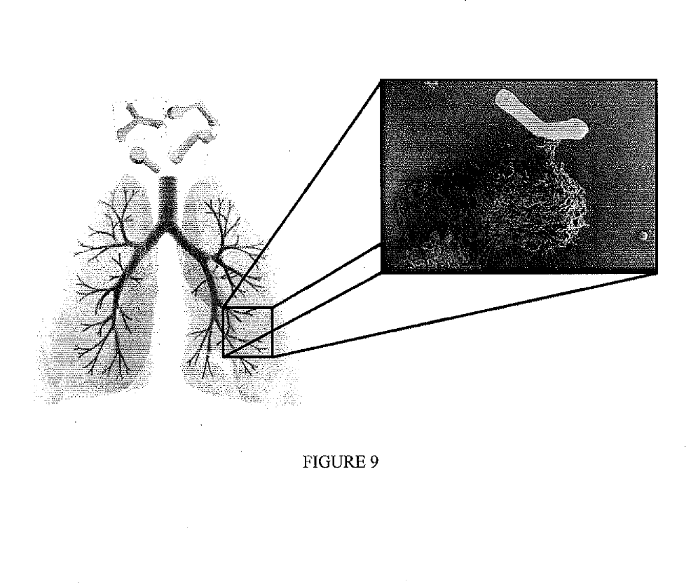

FIG. 10 depicts the motility of certain particles.

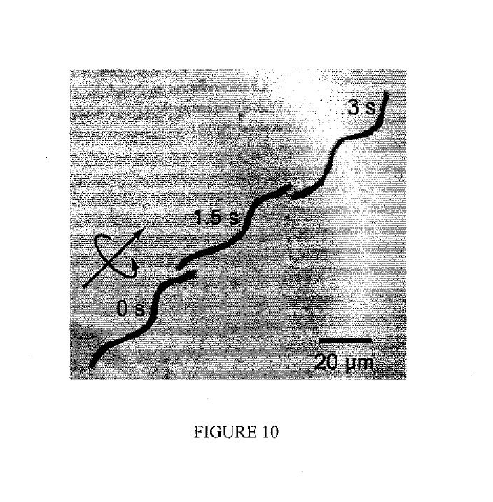

FIG. 11 depicts the shape diameter of two distinct particles.

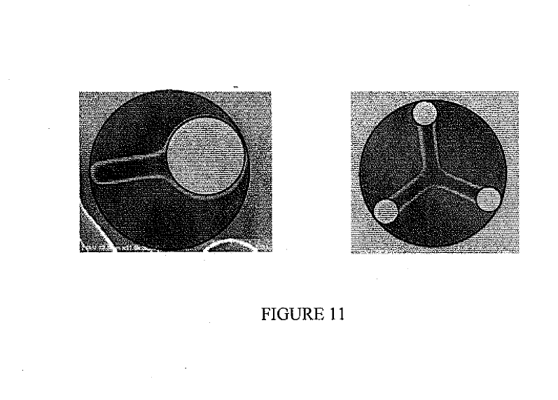

FIG. 12 depicts the trajectories of particles vis-à-vis particle geometry. From left to right: disk, donut, ring, button with three fenestrations, ellipsoid, fenestrated ellipsoid, Lorenz, fenestrated Lorenz, lollipop, pollen mimic, helicopter and v-dumbbell.

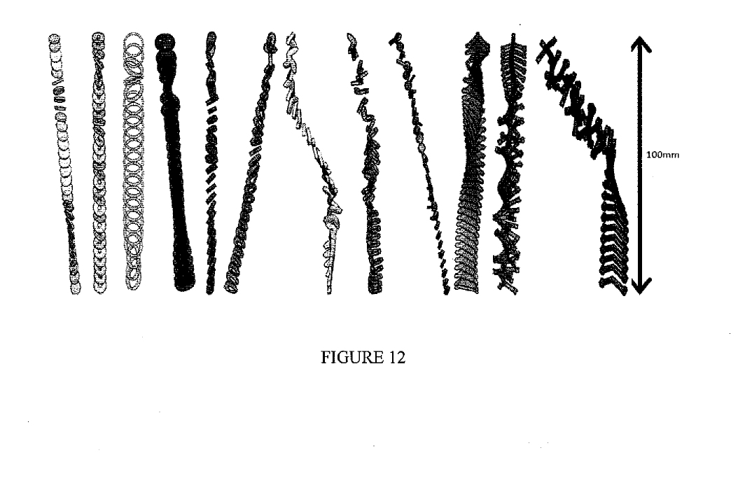

FIG. 13 depicts flow cytometry of cells gated into four populations: No particle association (no fluorescence; bottom left rectangle); membrane-bound particles only (red (R3 rectangle)); internalized particles only (green fluorescence (R6 rectangle)); membrane-bound and internalized particles (red and green fluorescence (R4 rectangle, red (left side, shown as grey) fading into green (right side, shown as light grey)).

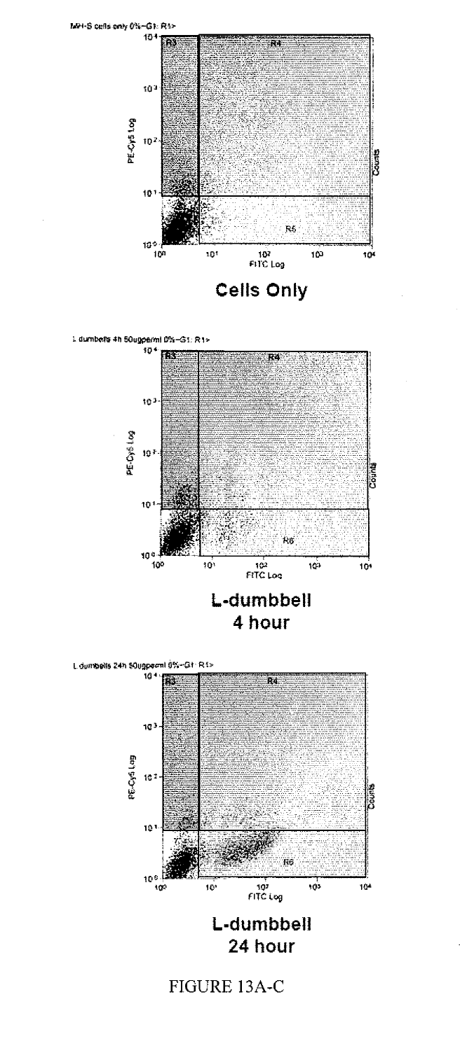

FIG. 14 depicts an over two-fold difference in alveolar macrophage phagocytosis of PRINT particles with similar MMADs. MMAD range: 2.05-3.35.

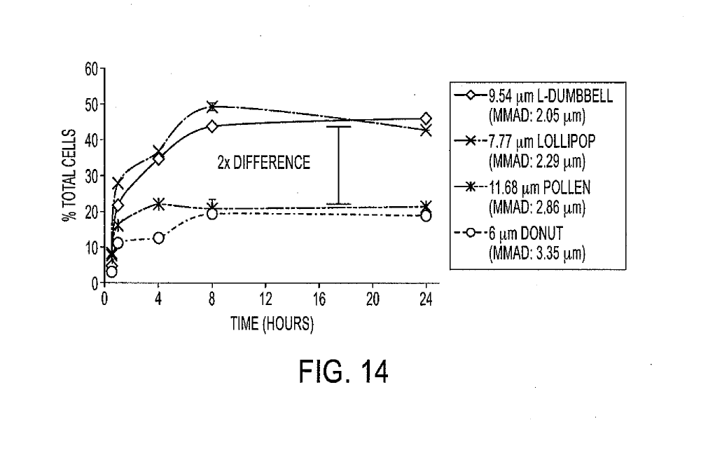

FIG. 15 shows MH-S cells dosed with 50 μg/ml helicopter PEG particles. Cells were fixed after one hour incubation with particles. Actin formation can differentiate between cell spreading and phagocytosis initiation. There is increased fluorescence in the actin (red (shown here in grey)) around the cell membrane which is currently internalizing the helicopter particles.

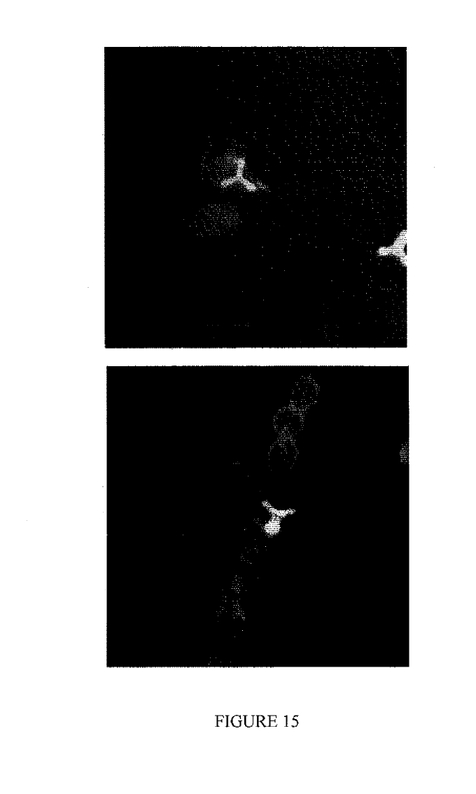

FIG. 16 shows MH-S cells dosed with 50 μg/ml helicopter PEG particles. Cells were fixed after 3 hours incubation with particles. There is increased fluorescence in the actin (red (shown here in grey)) around the cell membrane which is currently internalizing the helicopter particles.

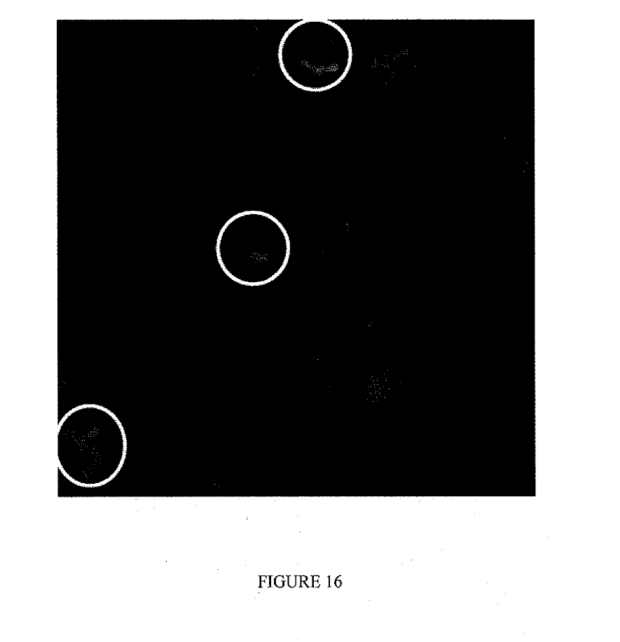

FIG. 17 shows actin localization in Calu-3 (human airway epithelial cells). Triton-X treatment removed the outer membrane making it possible to see actin network.

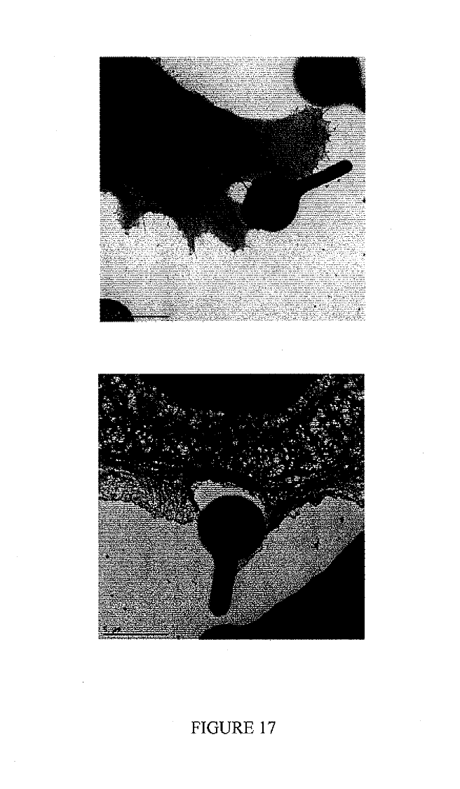

FIG. 18 shows actin localization in MH-S cells.

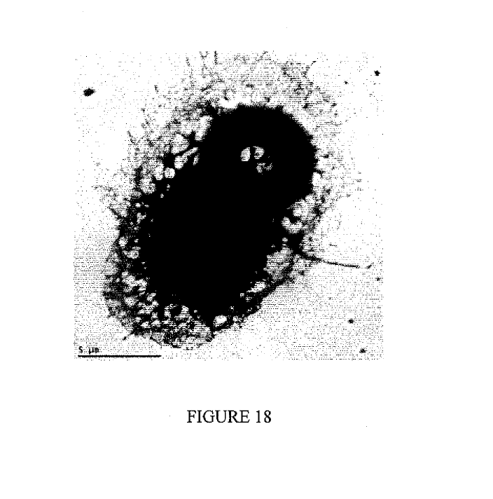

FIG. 19 depicts disease, drug, target and geometry of preferred pulmonary targets. The ability to guide the design of specific therapeutics based on cellular uptake and particle geometry provides a unique, systematic, rational design of therapeutics.

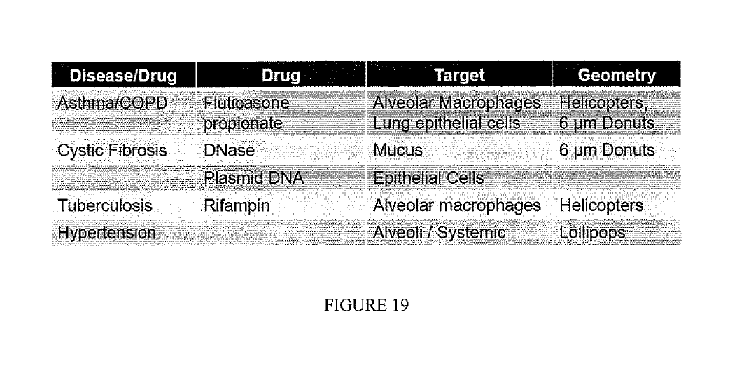

FIG. 20 depicts fabricated drug-loaded particles for specific pulmonary conditions.

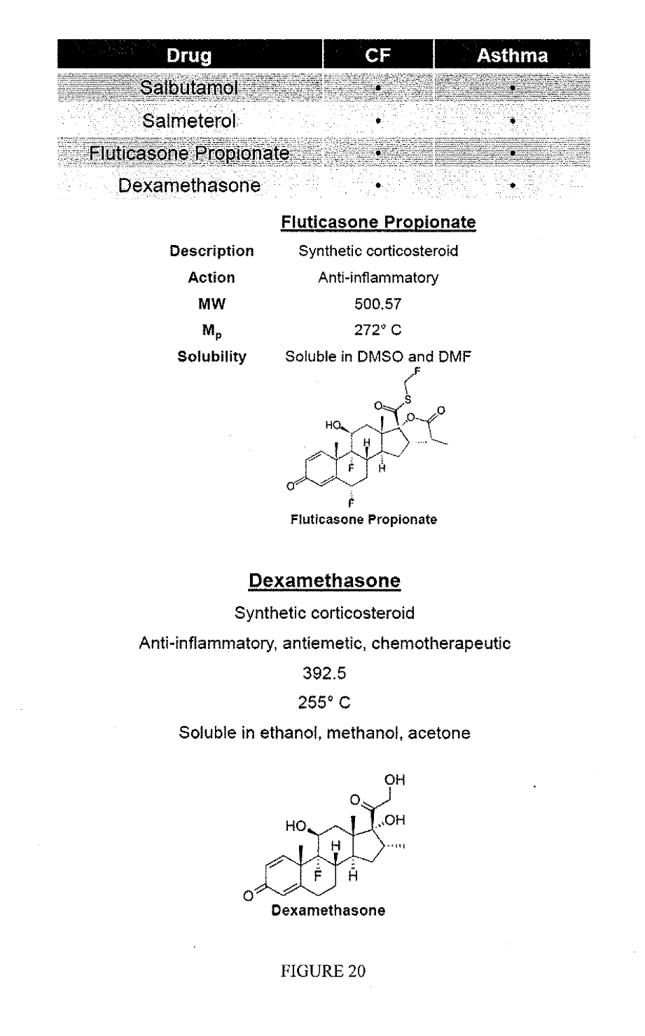

FIG. 21A-F shows particle orientation and kinetics of particle phagocytosis. Video analysis shows that particle orientation may rotate as the microphage's filopodia draw the particles towards the cell body. The time it takes to draw in the particles varies widely based on the particle distance from the main cell body.

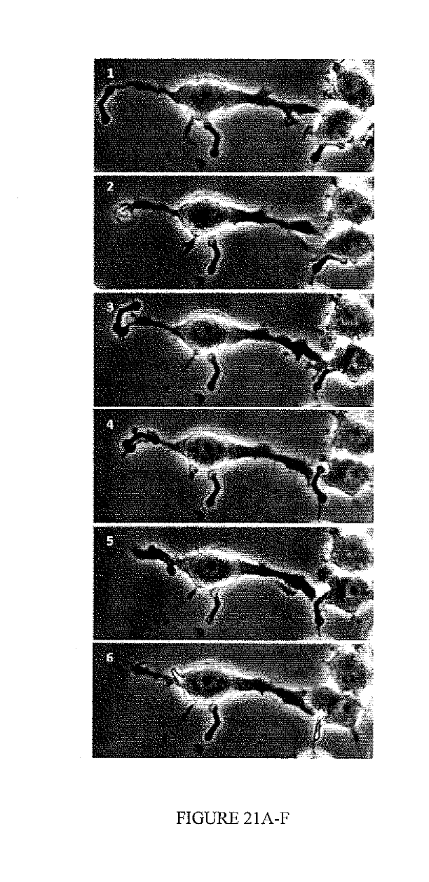

FIG. 22A-I shows particles fabricated from a variety of compositions: (A) BSA/lactose 200 nm×200 nm cylinders; (B) IgG/Lactose 10 μm pollen; (C) 30K PLGA 3 μm cylinders; (D) Itraconazole 1.5 μm donuts; (E) Itraconazole 3 μm donuts; (F) Itraconazole 6 μm donuts; (G) Zanamivir 1.5 μm donuts; (H) DNAse 1.5 μm donuts; (I) siRNA 1.5 μm donuts.

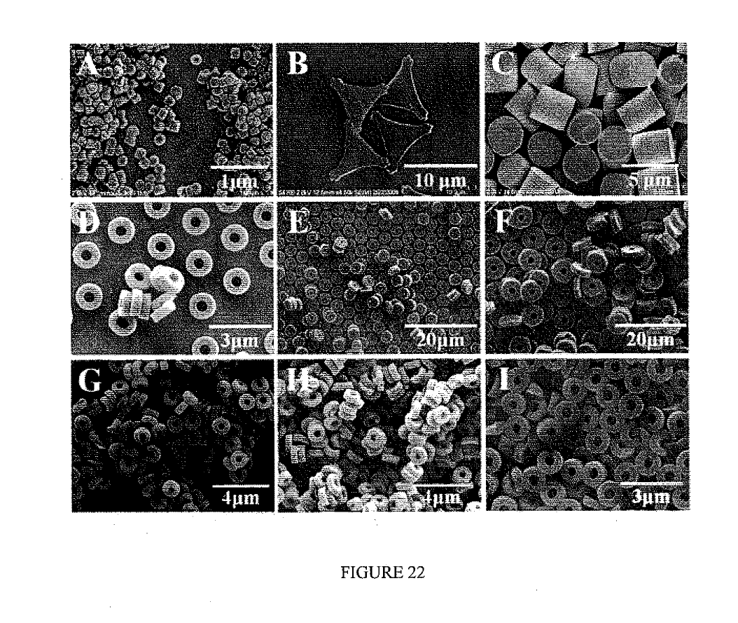

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims.

Disclosed herein are engineered particles that can be specifically tailored to possess certain geometries. It has now been found that different geometries can affect any immune response initiated upon in vivo administration of particles. In an embodiment, different shapes of particles can have a substantial impact on how an immune system responds to the particles. Data provided herein shows that different-shaped particles can have over two-fold difference in macrophage internalization though the particles have similar or substantially the same aerodynamic diameters (MMAD). The methods described herein provide a unique ability to geometrically tailor particles to modify delivery, residence time, immunological responses, etc. upon administration.

The term “agent,” “active agent,” or “drug” as used herein means any active pharmaceutical ingredient (“API”), including its pharmaceutically acceptable salts (e.g. the hydrochloride salts, the hydrobromide salts, the hydroiodide salts, and the saccharinate salts), as well as in the anhydrous, hydrated, and solvated forms, in the form of prodrugs, and in the individually optically active enantiomers of the API as well as polymorphs of the API. As discussed herein, the term “cargo” encompasses a drug or agent.

The amounts of cargo that can be incorporated into the polymer are substantially higher using the present method up to 100 wt. %. Also preferred are particles wherein the cargo comprises from about 1 wt. % to about 99 wt. % of the particle; from about 1 wt. % to about 98 wt. % of the particle; from about 1 wt. % to about 95 wt. % of the particle; from about 1 wt. % to about 90 wt. % of the particle; from about 1 wt. % to about 85 wt. % of the particle; from about 1 wt. % to about 80 wt. % of the particle; from about 1 wt. % to about 75 wt. % of the particle; from about 1 wt. % to about 50 wt. % of the particle; from about 1 wt. % to about 25 wt. % of the particle; from about 1 wt. % to about 10 wt. % of the particle; from about 10 wt. % to about 100 wt. % of the particle; from about 20 wt. % to about 100 wt. % of the particle; from about 30 wt. % to about 100 wt. % of the particle; from about 40 wt. % to about 100 wt. % of the particle; from about 50 wt. % to about 100 wt. % of the particle; from about 60 wt. % to about 100 wt. % of the particle; from about 70 wt. % to about 100 wt. % of the particle; from about 80 wt. % to about 100 wt. % of the particle; from about 85 wt. % to about 100 wt. % of the particle; from about 90 wt. % to about 100 wt. % of the particle; from about 95 wt. % to about 100 wt. % of the particle; from about 98 wt. % to about 100 wt. % of the particle; and from about 99 wt. % to about 100 wt. % of the particle.

Methods of preparing particles are described in US 2009/0028910; US 2009/0061152; WO 2007/024323; US 2009/0220789; US 2007/0264481; US 2010/0028994; US 2010/0196277; WO 2008/106503; US 2010/0151031; WO 2008/100304; WO 2009/041652; PCT/US 2010/041797; US 2008/0181958; WO 2009/111588; and WO 2009/132206, each of which is hereby incorporated by reference in their entirety. The particles are preferably molded wherein the molded particle further comprises a three-dimensional shape substantially mimicking the mold shape and a size less than about 50 micrometers in a broadest dimension. In further embodiments, the particles are preferably molded to have a three-dimensional shape substantially mimicking the mold shape and a size less than about 5 micrometers in a broadest dimension. Preferably, the molded particles have a first dimension of less than about 200 nanometers and a second dimension greater than about 200 nanometers.

In an embodiment, the present subject matter is directed to a composition comprising a plurality of substantially identically sized and shaped molded particles as described herein.

As used herein the term “mammal” refers to humans as well as all other mammalian animals. As used herein, the term “mammal” includes a “subject” or “patient” and refers to a warm blooded animal.

As used herein, the term “therapeutically effective” and “effective amount,” is defined as the amount of the pharmaceutical composition that produces at least some effect in treating a disease or a condition. For example, in a combination according to the invention, an effective amount is the amount required to inhibit the growth of cells of a neoplasm in vivo or an amount that can ameliorate symptoms of a pulmonary condition. The effective amount of active compound(s) used to practice the present invention for therapeutic treatment of neoplasms (e.g., cancer) varies depending upon the manner of administration, the age, body weight, and general health of the subject. It is within the skill in the art for an attending physician or veterinarian to determine the appropriate amount and dosage regimen. Such amounts may be referred to as “effective” amounts.

The term “MMAD” or “aerodynamic diameter” of a particle is used herein in accordance with its known meaning in the art. Methods of calculating the MMAD of a particle are known in the art and at least one method is disclosed herein.

Other particle characteristics used to describe the shapes examined include: a) the shape diameter (SD); it is the minimum diameter of a circumscribed circle around the particle; b) the minimum feature size (MFS); it is the diameter of the smallest distinct geometry of the shape; and c) the volume of the shape. All of these characteristics can be readily determined by one of skill in the art using the information disclosed herein and information known in the art.

As used herein, the term “spherical” or “substantially spherical” refers to a shape that is a sphere or is a natural shape such as an emulsion particle that resembles a sphere or a dispersion process that yields a spherical particle. A “non-spherical” shape does not include the spherical or substantially spherical shapes.

The term “amorphous” refers to a shape that is not engineered. A shape that is not prepared from a mold can be amorphous. Amorphous shapes by definition cannot be systematically reproducible. This is in contrast to molded shapes.

The term “hinders phagocytosis” or “reduced uptake by macrophages” refers to a slowing of the processes whereby an immune cell such as a macrophage internalizes a particle. Any inhibition at any point in the processes is encompassed by the term. Comparison of the rate of internalization can be performed by the methods disclosed herein and those methods that are known in the art. In one embodiment, the comparison is between a non-spherical engineered particle disclosed herein and a spherical particle having the same or substantially similar volume to the non-spherical engineered particle. In another embodiment, the comparison is between an engineered particle and a second engineered particle having one distinct feature as described herein. In this way, the desirable features can be identified. Methods of assessing uptake or phagocytosis are known in the art and at least one method is disclosed herein.

The term “macrophage uptake” refers to the process of internalization of an object by a macrophage. The process includes attachment of the macrophage to the object or immunological reaction of the macrophage towards the object through the process of phagocytosis and immunological neutralization or complete removal of the object by the macrophage.

As referred to herein, an amount or value that is the “same” or “substantially similar” is one that does not vary in a statistically significant way from a given reference point or value. With regard to particles formed by the present methods, the shapes and dimensions of the particles are reproducible and a plurality of particles is substantially identical. A plurality of particles means at least two particles. In embodiments, some insignificant artifacts may occur in some particles. In preferred embodiments, the particles are substantially identical. Scanning electron micrography can be used to evidence the substantially identical nature of the particles even at nanometer resolution. In all embodiments, the particles or a component(s) of the particle, such as an arm protruding from the body of the particle are configured and dimensioned to hinder phagocytosis of the particle by one or more macrophages.

The term “appendage” refers to any protrusion on the particle. This includes arms, branches, surface topography, ridges, etc. The appendage can further contain an appendage itself, for example, in the case of an arm that contains a branch. In embodiments, the appendage has a width to length ratio of greater than about 1:2. Preferably, the width to length ratio is greater than about 1:4. Preferably, the appendage protrudes at least about 4 micrometers in length from said body member. Also preferred are particles where the appendage has a total length of less than about 10 micrometers. More than one appendage can be present on a particle.

The term “pulmonary condition” refers to any disease or condition of the lungs and its associated tissues and structures. These conditions include, but are not limited to, cystic fibrosis, asthma, emphysema, tuberculosis, hypertension, interstitial lung disease (ILD), also known as diffuse parenchymal lung disease (DPLD), pulmonary inflammatory diseases, chronic obstructive pulmonary disease (COPD), allergic bronchopulmonary aspergillosis (ABPA), sarcoidosis, allergic rhinitis, bronchiectasis, pneumothorax, tumors, cysts, blebs, bullous diseases, etc. In embodiments, the particles and methods disclosed herein can be advantageously used to deliver an agent through a pulmonary airway such as a pulmonary bronchi or bronchiole.

As used herein, the term “substantially mimicking” means a molded particle that has a shape that is predetermined from the mold used to prepare the particle. This term includes variance in the shape, size, volume, etc. of the particle from the mold itself. However, the particles shape, size, volume etc. cannot be random since they are prepared from molds and substantially mimic the mold's shape, size, volume, etc.

Using PRINT, a top-down micro-molding particle fabrication technique, unique micron-sized particle geometries were prepared. As described fully elsewhere herein, the particles also may have a range of physical features such as fenestrations, angled arms, asymmetry and surface roughness, and charge which alter the interactions with cells and tissues.

As will be discussed fully elsewhere herein, aerodynamically shaped particles incorporating drugs and/or other biodegradable materials for pulmonary drug delivery, specifically to alveolar macrophages, and methods for their synthesis are provided. In a preferred embodiment, these shaped particles are made up of pure drug. Also preferred are particles that have a mass median aerodynamic diameter (MMAD) between about 1 μm and about 10 μm. Alternatively, the particles may be composed of therapeutic or diagnostic agents and additional materials to confer aerosolization and cellular uptake properties.

In these preferred embodiments, the particles are ideal as delivery vehicles for cargo intended to reach specific in vivo targets, tissues, organs, etc. More preferably, the particles are used to deliver pulmonary therapeutics into particular, targeted structures and tissues associated with the lungs. This avenue of delivery can also be used to deliver other systemic drugs.

In embodiments, the present subject matter is directed to rationally designed particles with varied shapes and sizes and surface charge which can incorporate drugs and/or other biomaterials for in vivo delivery. Preferably, the particles are designed with specific shape and/or size in order to target or de-target any immune response. In particular, the particles can be designed as described herein to target or de-target alveolar macrophages.

In embodiments, the particles described herein can be composed of pure therapeutic, drug and polymer, polymer only, protein, and protein plus therapeutic. The particles may also be composed of diagnostic agents and additional biomaterials to confer aerosolization and cellular uptake properties.

In another embodiment, the present subject matter is directed to a method of treating a mammal, comprising administering a particle or composition comprising a particle as disclosed herein, wherein the composition comprises a cargo, such as an agent or drug.

These additional embodiments are disclosed herein:

1. A method of preparing a particle having modified macrophage uptake comprising:

a. obtaining the calculated aerodynamic diameter of a first particle; and

b. preparing a second particle, wherein the volume of the second particle is different from that of said first particle and the calculated aerodynamic diameter is substantially the same as that of said first particle, and said second particle has an engineered shape;

wherein the macrophage uptake of the second particle is modified compared to the macrophage uptake of said first particle.

2. The method of embodiment 1, wherein said volume of the second particle is increased.

3. A particle, comprising:

an engineered geometry; and

a ratio of total volume (μm3) to calculated aerodynamic diameter of at least about 1.

4. The particle of embodiment 3, further comprising at least one fenestration, angled arm, surface topography or aerodynamic curve.

5. The particle of embodiment 3, wherein said geometry is asymmetric.

6. The particle of embodiment 3, wherein said calculated aerodynamic diameter is between about 0.1 μm to about 100 μm.

7. The particle of embodiment 3, having a calculated aerodynamic diameter between about 0.1 μm and about 10 μm.

8. The particle of embodiment 3, having a calculated aerodynamic diameter between about 0.5 μm and about 7 μm.

9. The particle of embodiment 3, having a calculated aerodynamic diameter between about 1 μm and about 5 μm.

10. The particle of embodiment 3, wherein said ratio is between about 1 and about 20.

11. The particle of embodiment 3, wherein said ratio is between about 2 and about 15.

12. The particle of embodiment 3, wherein said ratio is between about 3 and about 10.

13. The particle of embodiment 3, wherein said ratio is between about 10 and about 15.

14. The particle of embodiment 3, wherein said ratio is at least about 1.5.

15. The particle of embodiment 3, wherein said ratio is at least about 2.

16. The particle of embodiment 3, wherein said ratio is at least about 5.

17. The particle of embodiment 3, wherein said ratio is at least about 10.

18. The particle of embodiment 3, wherein said ratio is at least about 15.

19. The particle of embodiment 3, wherein said ratio is at least about 20.

20. The particle of embodiment 3, having a shape this is not substantially spherical.

21. The particle of embodiment 3, wherein said engineered geometry comprises at least one feature which hinders phagocytosis of said particle by a macrophage.

22. The particle of embodiment 21, wherein said feature is an appendage.

23. The particle of embodiment 17, wherein said feature is an arm.

24. The particle of embodiment 22, wherein said appendage contains at least one further appendage.

25. The particle of embodiment 21, wherein the size of said feature is greater than about 4 μm.

26. The particle of embodiment 3, comprising a polymer, a solution, a monomer, a plurality of monomers, a polymerization initiator, a polymerization catalyst, an inorganic precursor, a metal precursor, a pharmaceutical agent, a tag, a magnetic material, a paramagnetic material, a superparamagnetic material, a ligand, a cell penetrating peptide, a porogen, a surfactant, a plurality of immiscible liquids, a solvent or a charged species.

27. The particle of embodiment 3, wherein said engineered geometry is substantially a toroid-shape.

28. The particle of embodiment 3, wherein said engineered geometry is substantially a ball-and-stick shape.

29. The particle of embodiment 3, wherein said engineered geometry is substantially a helicopter shape.

30. The particle of embodiment 3, wherein said engineered geometry is substantially a pollen-shape.

31. The particle of embodiment 3, wherein said engineered geometry is substantially a dumbbell-shape.

32. The particle of embodiment 3, wherein said engineered geometry is substantially a boomerang-shape.

33. A method of delivering an agent comprising,

administering the engineered particle of embodiment 3, wherein said particle comprises said agent, and wherein said particle exhibits reduced uptake by macrophages compared to a substantially spherical particle having substantially the same volume as said engineered particle.

34. A method for controlled release of an agent comprising,

administering particles of embodiment 3, wherein said particles comprise said agent and are resistant to phagocytosis, wherein at least about 50% of said particles have not been phagocytized at 24 hours after said administration.

35. The method of embodiment 34, wherein at least about 60% of said particles have not been phagocytized at 24 hours after said administration.

36. The method of embodiment 34, wherein at least about 70% of said particles have not been phagocytized at 24 hours after said administration.

37. A method of selecting internalization kinetics of a particle comprising,

a. assessing internalization kinetics of a particle by a macrophage, wherein said particle has a feature,

b. assessing internalization kinetics of a second particle by a macrophage, wherein said second particle has at least one distinct feature;

c. comparing the internalization kinetics in a and b; and

d. preparing an engineered particle based on the comparison in c.

38. The method of embodiment 37, wherein

b. further comprises assessing the internalization kinetics of additional particles, wherein each particle has at least one distinct feature, and

c. further comprises comparing the internalization kinetics of all particles.

39. A composition comprising,

a first particle having an engineered shape; and

a second particle having a natural or engineered shape that is different from said first particle;

wherein the macrophage uptake of said first particle is reduced compared to that of said second particle.

40. The composition of embodiment 39, wherein said first particle comprises a

an engineered geometry; and

a ratio of total volume (μm3) to calculated aerodynamic diameter of at least about 1.

41. The composition of embodiment 40, wherein said ratio is between about 1 and about 20.

42. The composition of embodiment 40, wherein said ratio is between about 2 and about 15.

43. The composition of embodiment 40, wherein said ratio is between about 3 and about 10.

44. The composition of embodiment 40, wherein said ratio is between about 10 and about 15.

45. The composition of embodiment 40, wherein said ratio is at least about 1.5.

46. The composition of embodiment 40, wherein said ratio is at least about 2.

47. The composition of embodiment 40, wherein said ratio is at least about 5.

48. The composition of embodiment 40, wherein said ratio is at least about 10.

49. The composition of embodiment 40, wherein said ratio is at least about 15.

50. The composition of embodiment 40, wherein said ratio is at least about 20.

51. The composition of embodiment 39, wherein said first particle has a calculated aerodynamic diameter between about 0.1 μm to about 100 μm.

52. The composition of embodiment 39, wherein said first particle has a calculated aerodynamic diameter between about 0.1 μm and about 10 μm.

53. The composition of embodiment 39, wherein said first particle has a calculated aerodynamic diameter between about 0.5 μm and about 7 μm.

54. The composition of embodiment 39, wherein said first particle has a calculated aerodynamic diameter between about 1 μm and about 5 μm.

55. The composition of embodiment 39, wherein said composition is an aerosol.

56. The composition of embodiment 39, wherein said second particle has a natural shape.

57. The composition of embodiment 39, wherein said second particle has a substantially spherical or amorphous shape.

58. A drug delivery device, comprising:

a plurality of particles each having a substantially similar engineered geometry, wherein the engineered geometry is configured and dimensioned to hinder phagocytosis by a macrophage.

59. A drug delivery device that exhibits reduced uptake by macrophages, comprising:

a particle having an engineered geometry, wherein said engineered geometry is configured and dimensioned to hinder phagocytosis by a macrophage.

60. The drug delivery device of embodiment 59, wherein the macrophage is an alveolar macrophage.

61. The drug delivery device of embodiment 59, wherein the engineered geometry comprises a body member and an appendage protruding from the body member.

62. The drug delivery device of embodiment 61, wherein the appendage comprises a width to length ratio of greater than 1:2.

63. The drug delivery device of embodiment 61, wherein the appendage has a width to length ratio of greater than 1:4.

64. The drug delivery device of embodiment 61, wherein the appendage protrudes at least about 4 micrometers in length from the body member.

65. The drug delivery device of embodiment 61, further comprising a second appendage protruding from said body member.

66. The drug delivery device of embodiment 59, wherein the particle comprises a polymer, a monomer, a plurality of monomers, a polymerization initiator, a polymerization catalyst, an inorganic precursor, a metal precursor, a pharmaceutical agent, a tag, a magnetic material, a paramagnetic material, a superparamagnetic material, a ligand, a cell penetrating peptide, a porogen, a surfactant, a charged species, or a biologic.

67. The drug delivery device of embodiment 59, wherein the engineered geometry is substantially a toroid-shape, substantially a ball-and-stick shape, substantially a helicopter shape, substantially a pollen-shape, substantially a dumbbell-shape, or substantially a boomerang-shape.

68. The drug delivery device of embodiment 59, wherein the particle has a ratio of total volume to calculated aerodynamic diameter of at least about 1.

69. The drug delivery device of embodiment 58, wherein the ratio is between about 1 and about 20.

70. A method of hindering phagocytosis of an agent by a macrophage comprising,

administering a plurality of particles wherein each particle is configured and dimensioned with an engineered geometry and comprises an agent or drug, wherein said engineered geometry exhibits reduced uptake by macrophages compared to a substantially spherical particle having substantially the same volume as the engineered particle.

71. The method of embodiment 70, wherein at least about 50% of the particles have not been phagocytized by a macrophage at about 24 hours after said administration.

72. The method of embodiment 70, wherein at least about 60% of the particles have not been phagocytized by a macrophage at about 24 hours after said administration.

73. The method of embodiment 70, wherein the engineered geometry comprises a body member and an appendage protruding from the body member, wherein the appendage is configured with a width to length ratio of greater than about 1:2, wherein the agent is released from said appendage.

74. The method of embodiment 73, wherein the macrophage is an alveolar macrophage.

75. A method of selecting internalization kinetics of a particle comprising,

a. assessing internalization kinetics of a particle by a macrophage, wherein the particle has a feature,

b. assessing internalization kinetics of a second particle by a macrophage, wherein the second particle has at least one distinct feature;

c. comparing the internalization kinetics in a and b; and

d. preparing an engineered particle based on the comparison in c.

76. The method of embodiment 75, wherein b. further comprises assessing the internalization kinetics of additional particles, wherein each particle has at least one distinct feature, and c. further comprises comparing the internalization kinetics of all particles.

77. The drug delivery device of embodiment 59, further comprising an agent or drug.

78. The drug delivery device of embodiment 59, wherein said particles are engineered for lung deposition and reduced uptake by macrophages.

i. Pulmonary Delivery

The ability to enhance or decrease macrophage uptake for drug delivery applications is an important avenue to explore for pulmonary therapeutics. As described herein, different geometries of aerodynamically shaped particles can influence the extent of alveolar macrophage phagocytosis. For example, 6 μm torus particles may be used to avoid alveolar macrophage uptake and clearance while 1.5 μm torus particles may be used to enhance alveolar macrophage and deep lung deposition. Thus, the geometries that effect therapeutic efficacy as well as macrophage uptake can be identified and efficiently controlled. Additionally, having the ability to target or de-target pulmonary macrophages upon administration of aerosolized drug particles will be a powerful tool in the design of inhaled drug delivery systems.

As a review of the air conducting system of the lung, after entering the nose and/or mouth, air moves through the pharynx and larynx into the trachea and through the bronchi and bronchioles until ultimately entering the alveoli where gas exchange between blood and the atmosphere occurs. In humans, the trachea bifurcates at the carina into a right main bronchus in the right lung and a left main bronchus in the left lung. Each main bronchi divides into secondary or lobar bronchi (two on the left, three on the right) which supply a lobe of the lung. Each lobar bronchus further divides into tertiary (segmental) bronchi which supply specific bronchopulmonary segments. Within each segment there is further branching of the bronchi into bronchioles (conducting, terminal and respiratory bronchioles) which lead to the alveolar ducts and sacs. Pulmonary blood vessels (i.e., pulmonary and bronchial arteries and veins) carry blood to and from the lungs and follow paths adjacent the pulmonary airways. Generally, the trachea and proximal bronchi comprise hyaline type cartilage which transitions into an elastic cartilage in the smaller airways and ultimately to smooth muscle closer to the alveoli. An elastic connective tissue frame work surrounding the airways and blood vessels enables the lungs to expand and contract during respiration. Aerosols of solid particles comprising the anti-malarial compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the anti-malarial compound, a suitable powder diluent, such as lactose, and an optional surfactant. A second type of illustrative aerosol generator comprises a metered dose inhaler. Metered dose inhalers are pressurized aerosol dispensers, typically containing a suspension or solution formulation of the anti-malarial compound in a liquified propellant. During use these devices discharge the formulation through a valve, adapted to deliver a metered volume, from 10 to 22 microliters to produce a fine particle spray containing the anti-malarial compound.

Suitable propellants include certain chlorofluorocarbon (compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents. Any propellant may be used in carrying out the present invention, including both chlorofluorocarbon-containing propellants and non-chlorofluorocarbon-containing propellants. Fluorocarbon aerosol propellants that may be employed in carrying out the present invention including fluorocarbon propellants in which all hydrogen are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. A stabilizer such as a fluoropolymer may optionally be included in formulations of fluorocarbon propellants, such as described in U.S. Pat. No. 5,376,359 to Johnson.

The present invention relates generally to specifically shaped particles with MMAD's within the pulmonary deposition range for use in drug delivery to the lung and specifically to target or de-target pulmonary macrophages. The ease of accessibility and the large surface area of the lung make it an attractive target for both local and systemic drug delivery. It has been shown that the shape and porosity of aerosol particles can determine the area of sedimentation in the lung, thus, having the ability to modulate particle features will help maximize drug dispersion to the optimal therapeutic area. The development of the PRINT (Particle Replication in Non-wetting Templates) process allows for the elucidation of particle shape and size in drug delivery applications by demonstrating the ability to specifically alter one characteristic variable at a time.

The engineered approach to these particles gives precise control over many particle characteristics such as size, shape, composition, porosity, modulus, and surface functionality (1, 2). Thus, the influence of particle geometry on cellular internalization of macromolecules and nanoparticles can be more extensively explored using this technology.

Cellular internalization and intracellular trafficking of particles may be influenced by particle geometry (3, 4). Shape effects on macrophage phagocytosis of polystyrene particles in vitro have found that the local contact angle may play a role in the initiation of phagocytosis (4, 5). Large aspect ratio particles have also been shown to affect macrophage uptake (6). As disclosed herein, PRINT, a top-down micro-molding method, was utilized to manufacture unique geometric shapes with varying numbers of appendages and end terminal shapes and sizes. The shapes and sizes can affect macrophage phagocytosis.

Because of the amenable fabrication conditions using PRINT, particles composed of various polymers, chemotherapeutics, and sensitive biologics can be produced leading to direct therapeutic application (7, 8). Therapies targeting macrophages vary widely in their exploitation of the cell's sequestering, signaling, and homing abilities.

In pulmonary delivery in particular, therapeutics must circumvent the lung's particle clearance mechanisms such as mucociliary transport, phagocytosis by macrophages (9), and rapid absorption of drug molecules into the systemic circulation (10). Mucociliary clearance can be reduced by avoiding particle deposition in the tracheobronchial region which contains the cilia and goblets cells that make up the mucociliary escalator (11). Upon delivery to the pulmonary region, particles are rapidly cleared by alveolar macrophages (AM) (12).

The sequestering of bacteria in macrophages for diseases such as tuberculosis (TB) and pneumonia may make AMs prime targets for antibacterial delivery (13) and targeted apoptosis of AMs has also been shown to lead to increased protection from M. tuberculosis infection and an enhanced TH1-mediated immune response (14). However, avoidance of macrophage uptake may be preferred for aerosolized therapeutics targeted to alveoli and epithelial cells as well as for systemic delivery (15). This was shown in studies in which depletion of AMs by clodronate resulted in an increased pulmonary bioavailability of larger inhaled proteins such as IgG and human chorionic gonadotropin (hCG) (12). Particles with geometric diameters of 1-5 microns have been shown to be preferentially taken up by AMs (15, 16). Relatively large, porous particles were developed to deposit in the alveolar airspace and successfully avoid

macrophage uptake due to their large (>5 μm) geometric size (15).

ii. Particles

The particles may be formed of pure drug and/or additional materials such as biodegradable polymers, proteins, or other water soluble or non-water soluble materials may be incorporated into the particle. Shaped particles of similar total volumes incorporating varying numbers and sizes of appendages were fabricated using PRINT™ technology (U.S. Publication No. 2009/0028910 to DeSimone et al., filed Dec. 20, 2004). Four particle characteristics are used to describe the shapes examined: the shape diameter (SD) is the minimum diameter of a circumscribed circle around the particle; the minimum feature size (MFS) is the diameter of the smallest distinct geometry of the shape; the volume of the shape; and the aerodynamic diameter (MMAD) of the shape (Table 1).

Table 1 is a table of fabricated shapes, their dimensions, and their measured MMAD as measured by an Aerosol Particle Sizer (APS).

TABLE 1
Particle Geometries

| | Total | Surface | Minimum | MMAD |
|---|---|---|---|---|
| | Volume | Area/side | Feature | (calculated) |
| Shape | (μm²) | (μm²) | Size (μm²⁾ | (μm) |
| 1.5 μm donut | 0.79 | 1.57 | 1.5 | — |
| 3 μm donut | 5.03 | 6.28 | 3 | 1.78 |
| 10.24 μm helicopter | 20.91 | 15.72 | 1.6 | 1.86 |
| 12.3 μm V-boomerang | 21.73 | 16.34 | 2.25 | 1.99 |
| 9.54 μm L-dumbbell | 20.64 | 15.52 | 2.5 | 2.05 |
| 7.77 μm Lollipop | 21.67 | 16.29 | 1 | 2.29 |
| 11.68 μm Pollen | 35.60 | 50.86 | 10 | 2.86 |
| 6 μm donut | 43.98 | 25.87 | 6 | 3.35 |

The particle itself can be fabricated using a polymer. Preferably, the polymer is a water soluble polymer. More preferably, the polymer is a PEG, PLGA, PMMA, or other biocompatible, biodegradable, or the like polymer. Table 2 shows a representative particle composition.

TABLE 2
Particle Composition

| Component | Weight % |
|---|---|
| F—O—A (Fluorescein-O-acrylate) | 2% |
| DEAP (2,2-diethoxyacetophenone) | 2% |
| PEG₇₀₀-DA (poly(ethylene glycol) diacrylate) | 96% |

In some embodiments, the polymer is “PEG” or “poly(ethylene glycol)” as used herein, is meant to encompass any water-soluble poly(ethylene oxide). Typically, PEGs for use in the present invention will comprise the following structure: “—(CH₂CH₂O)ₙ—”. The variable (n) is 3 to 3,000, or about 3 to about 30,000; about 3 to about 10,000 or about 3 to about 5,000. The terminal groups and architecture of the overall PEG may vary. PEGs having a variety of molecular weights, structures or geometries as is known in the art. “Water-soluble”, in the context of a water soluble polymer is any segment or polymer that is soluble in water at room temperature. Typically, a water-soluble polymer or segment will transmit at least about 75%, more preferably at least about 95% of light, transmitted by the same solution after filtering. On a weight basis, a water-soluble polymer or segment thereof will preferably be at least about 35% (by weight) soluble in water, more preferably at least about 50% (by weight) soluble in water, still more preferably about 70% (by weight) soluble in water, and still more preferably about 85% (by weight) soluble in water. It is most preferred, however, that the water-soluble polymer or segment is about 95% (by weight) soluble in water or completely soluble in water.

An “end-capping” or “end-capped” group is an inert group present on a terminus of a polymer such as PEG. An end-capping group is one that does not readily undergo chemical transformation under typical synthetic reaction conditions. An end capping group is generally an alkoxy group, —OR, where R is an organic radical comprised of 1-20 carbons and is preferably lower alkyl (e.g., methyl, ethyl) or benzyl. “R” may be saturated or unsaturated, and includes aryl, heteroaryl, cyclo, heterocyclo, and substituted forms of any of the foregoing. When the polymer has an end-capping group comprising a detectable label, the amount or location of the polymer and/or the moiety (e.g., active agent) to which the polymer is coupled, can be determined by using a suitable detector. Such labels include, without limitation, fluorescers, chemiluminescers, moieties used in enzyme labeling, calorimetric (e.g., dyes), metal ions, radioactive moieties, and the like.

As used herein, the term “tracers” include, without limitation, fluorescers, chemiluminescers, moieties used in enzyme labeling, calorimetric (e.g., dyes), metal ions, radioactive moieties, and the like.

The cargo contained in the particle can be selected from the group consisting of analgesics, anti-cancer agents, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opioid analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof. More preferably, the pharmaceutical agent is an anti-cancer agent. It is also preferred that the pharmaceutical or biological agent is selected from quinoline alkaloids, taxanes, anthracyclines, nucleosides, kinase inhibitors, tyrosine kinase inhibitors, antifolates, proteins and nucleic acids. More preferably, the pharmaceutical or biological agent is selected from the group consisting of Camptothecin, Topotecan, Irinotecan, SN-38, Paclitaxel, Docetaxel, Daunorubicin, Doxorubicin, Epirubicin, Idarubicin Gemcitabine, Cytarabine, Brefeldin-A Imatinib, Gefitinib, Lapatinib, Sunitinib, Methotrexate, Folinic Acid, Efflux Inhibitors, ATP-Binding Inhibitors, Cytochrome-C, Ovalbumin, siRNA Anti-Luciferase, siRNA Androgen Receptor and RNA Replicon. Biological agents are preferably DNA, RNA, siRNA, cDNA, proteins or immunoglobulins. Useful chemical agents include a pesticide, fungicide, insecticide, herbicide or biocide.

iii. Additional Routes of Administration

In an embodiment, the subject matter disclosed herein comprises administering to a subject a therapeutically effective amount of a particle described herein. Routes of administration for a therapeutically effective amount of a particle composition or delivery vehicle include but are not limited to intravenous or parenteral administration, oral administration, topical administration, transmucosal administration and transdermal administration. For intravenous or parenteral administration, i.e., injection or infusion, the composition may also contain suitable pharmaceutical diluents and carriers, such as water, saline, dextrose solutions, fructose solutions, ethanol, or oils of animal, vegetative, or synthetic origin. It may also contain preservatives, and buffers as are known in the art.

When a therapeutically effective amount is administered by intravenous, cutaneous or subcutaneous injection, the solution can also contain components to adjust pH, isotonicity, stability, and the like, all of which is within the skill in the art. The pharmaceutical composition of the present invention may also contain stabilizers, preservatives, buffers, antioxidants, or other additive known to those of skill in the art. Typically, compositions for intravenous or parenteral administration comprise a suitable sterile solvent, which may be an isotonic aqueous buffer or pharmaceutically acceptable organic solvent. The compositions can also include a solubilizing agent as is known in the art if necessary. Compositions for intravenous or parenteral administration can optionally include a local anesthetic to lessen pain at the site of the injection. Generally, the ingredients are supplied either separately or mixed together in unit dosage form in a hermetically sealed container such as an ampoule or sachette. The pharmaceutical compositions for administration by injection or infusion can be dispensed, for example, with an infusion bottle containing, for example, sterile pharmaceutical grade water or saline. Where the pharmaceutical compositions are administered by injection, an ampoule of sterile water for injection, saline, or other solvent such as a pharmaceutically acceptable organic solvent can be provided so that the ingredients can be mixed prior to administration.

The duration of intravenous therapy using the pharmaceutical composition of the present invention will vary, depending on the condition being treated or ameliorated and the condition and potential idiosyncratic response of each individual mammal. The duration of each infusion is from about 1 minute to about 1 hour. The infusion can be repeated as necessary.

Systemic formulations include those designed for administration by injection, e.g., subcutaneous, intravenous, intramuscular, intrathecal or intraperitoneal injection. Useful injectable preparations include sterile suspensions, solutions or emulsions of the active compound(s) in aqueous or oily vehicles. The compositions also can contain solubilizing agents, formulating agents, such as suspending, stabilizing and/or dispersing agent. The formulations for injection can be presented in unit dosage form, e.g., in ampules or in multidose containers, and can contain added preservatives. For prophylactic administration, the compound can be administered to a patient at risk of developing one of the previously described conditions or diseases. Alternatively, prophylactic administration can be applied to avoid the onset of symptoms in a patient suffering from or formally diagnosed with the underlying condition.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein.

Oral administration of the composition or vehicle can be accomplished using dosage forms including but not limited to capsules, caplets, solutions, suspensions and/or syrups. Such dosage forms are prepared using conventional methods known to those in the field of pharmaceutical formulation and described in the pertinent texts, e.g., in Remington: The Science and Practice of Pharmacy (2000), supra.

The dosage form may be a capsule, in which case the active agent-containing composition may be encapsulated in the form of a liquid. Suitable capsules may be either hard or soft, and are generally made of gelatin, starch, or a cellulosic material, with gelatin capsules preferred. Two-piece hard gelatin capsules are preferably sealed, such as with gelatin bands or the like. See, for e.g., Remington: The Science and Practice of Pharmacy (2000), supra, which describes materials and methods for preparing encapsulated pharmaceuticals.

Capsules may, if desired, be coated so as to provide for delayed release. Dosage forms with delayed release coatings may be manufactured using standard coating procedures and equipment. Such procedures are known to those skilled in the art and described in the pertinent texts (see, for e.g., Remington: The Science and Practice of Pharmacy (2000), supra). Generally, after preparation of the capsule, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained-release dosage forms provide for drug release over an extended time period, and may or may not be delayed release. Generally, as will be appreciated by those of ordinary skill in the art, sustained-release dosage forms are formulated by dispersing a drug within a matrix of a gradually bioerodible (hydrolyzable) material such as an insoluble plastic, a hydrophilic polymer, or a fatty compound. Insoluble plastic matrices may be comprised of for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinyl acetate phthalate, vinylacetate crotonic acid copolymer, and ethylene-vinyl acetate copolymers; zein; and shellac, ammoniated shellac, shellac-acetyl alcohol, and shellac n-butyl stearate, Fatty compounds for use as a sustained release matrix material include, but are not limited to, waxes generally (e.g., carnauba wax) and glyceryl tristearate.

Topical administration of a particle or delivery vehicle can be accomplished using any formulation suitable for application to the body surface, and may comprise, for example, an ointment, cream, gel, lotion, solution, paste or the like, and/or may be prepared so as to contain liposomes, micelles, and/or microspheres. Preferred topical formulations herein are ointments, creams, and gels.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy (2000), supra, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight (See, e.g., Remington: The Science and Practice of Pharmacy (2002), supra),

Creams, as also well known in the art, are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels-are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methylcellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

Various additives, known to those skilled in the art, may be included in the topical formulations. For example, solubilizers may be used to solubilize certain active agents. For those drugs having an unusually low rate of permeation through the skin or mucosal tissue, it may be desirable to include a permeation enhancer in the formulation; suitable enhancers are as described elsewhere herein.

Transmucosal administration of a particle composition or delivery vehicle can be accomplished using any type of formulation or dosage unit suitable for application to mucosal tissue. For example, a particle composition or delivery vehicle may be administered to the buccal mucosa in an adhesive patch, sublingually or lingually as a cream, ointment, or paste, nasally as droplets or a nasal spray, or by inhalation of an aerosol formulation or a non-aerosol liquid formulation.

Preferred buccal dosage forms will typically comprise a therapeutically effective amount of a particle composition and a bioerodible (hydrolyzable) polymeric carrier that may also serve to adhere the dosage form to the buccal mucosa. The buccal dosage unit is fabricated so as to erode over a predetermined time period, wherein drug delivery is provided essentially throughout. The time period is typically in the range of from about 1 hour to about 72 hours. Preferred buccal delivery preferably occurs over a time period of from about 2 hours to about 24 hours. Buccal drug delivery for short-term use should preferably occur over a time period of from about 2 hours to about 8 hours, more preferably over a time period of from about 3 hours to about 4 hours. As needed buccal drug delivery preferably will occur over a time period of from about 1 hour to about 12 hours, more preferably from about 2 hours to about 8 hours, most preferably from about 3 hours to about 6 hours. Sustained buccal drug delivery will preferably occur over a time period of from about 6 hours to about 72 hours, more preferably from about 12 hours to about 48 hours, most preferably from about 24 hours to about 48 hours. Buccal drug delivery, as will be appreciated by those skilled in the art, avoids the disadvantages encountered with oral drug administration, e.g., slow absorption, degradation of the active agent by fluids present in the gastrointestinal tract and/or first-pass inactivation in the liver.

The “therapeutically effective amount” of a particle composition or delivery vehicle in the buccal dosage unit will of course depend on the potency and the intended dosage, which, in turn, is dependent on the particular individual undergoing treatment, the specific indication, and the like. The buccal dosage unit will generally contain from about 1.0 wt. % to about 60 wt. % active agent, preferably on the order of from about 1 wt. % to about 30 wt, % active agent. With regard to the bioerodible (hydrolyzable) polymeric carrier, it will be appreciated that virtually any such carrier can be used, so long as the desired drug release profile is not compromised, and the carrier is compatible with the particle composition or delivery vehicle and any other components of the buccal dosage unit. Generally, the polymeric carrier comprises a hydrophilic (water-soluble and water-swellable) polymer that adheres to the wet surface of the buccal mucosa. Examples of polymeric carriers useful herein include acrylic acid polymers and co, e.g., those known as “carbomers” (Carbopol®, which may be obtained from B. F. Goodrich, is one such polymer). Other suitable polymers include, but are not limited to: hydrolyzed polyvinylalcohol; polyethylene oxides (e.g., Sentry Polyox® water soluble resins, available from Union Carbide); polyacrylates (e.g., Gantrez®, which may be obtained from GAF); vinyl polymers and copolymers; polyvinylpyrrolidone; dextran; guar gum; pectins; starches; and cellulosic polymers such as hydroxypropyl methylcellulose, (e.g., Methocel®, which may be obtained from the Dow Chemical Company), hydroxypropyl cellulose (e.g., Klucel®, which may also be obtained from Dow), hydroxypropyl cellulose ethers (see, e.g., U.S. Pat. No. 4,704,285 to Alderman), hydroxyethyl cellulose, carboxymethyl cellulose, sodium carboxymethyl cellulose, methyl cellulose, ethyl cellulose, cellulose acetate phthalate, cellulose acetate butyrate, and the like.

Other components may also be incorporated into the buccal dosage forms described herein. The additional components include, but are not limited to, disintegrants, diluents, binders, lubricants, flavoring, colorants, preservatives, and the like. Examples of disintegrants that may be used include, but are not limited to, cross-linked polyvinylpyrrolidones, such as crospovidone (e.g., Polyplasdone® XL, which may be obtained from GAF), cross-linked carboxylic methylcelluloses, such as croscarmellose (e.g., Ac-di-sol®, which may be obtained from FMC), alginic acid, and sodium carboxymethyl starches (e.g., Explotab®, which may be obtained from Edward Medell Co., Inc.), methylcellulose, agar bentonite and alginic acid. Suitable diluents are those which are generally useful in pharmaceutical formulations prepared using compression techniques, e.g., dicalcium phosphate dihydrate (e.g., Di-Tab®, which may be obtained from Stauffer), sugars that have been processed by cocrystallization with dextrin (e.g., co-crystallized sucrose and dextrin such as Di-Pak®, which may be obtained from Amstar), calcium phosphate, cellulose, kaolin, mannitol, sodium chloride, dry starch, powdered sugar and the like. Binders, if used, are those that enhance adhesion. Examples of such binders include, but are not limited to, starch, gelatin and sugars such as sucrose, dextrose, molasses, and lactose. Particularly preferred lubricants are stearates and stearic acid, and an optimal lubricant is magnesium stearate.

Sublingual and lingual dosage forms include creams, ointments and pastes. The cream, ointment or paste for sublingual or lingual delivery comprises a therapeutically effective amount of the selected active agent and one or more conventional nontoxic carriers suitable for sublingual or lingual drug administration. The sublingual and lingual dosage forms of the present invention can be manufactured using conventional processes. The sublingual and lingual dosage units are fabricated to disintegrate rapidly. The time period for complete disintegration of the dosage unit is typically in the range of from about 10 seconds to about 30 minutes, and optimally is less than 5 minutes.

Other components may also be incorporated into the sublingual and lingual dosage forms described herein. The additional components include, but are not limited to binders, disintegrants, wetting agents, lubricants, and the like. Examples of binders that may be used include water, ethanol, polyvinylpyrrolidone; starch solution gelatin solution, and the like. Suitable disintegrants include dry starch, calcium carbonate, polyoxyethylene sorbitan fatty acid esters, sodium lauryl sulfate, stearic monoglyceride, lactose, and the like. Wetting agents, if used, include glycerin, starches, and the like. Particularly preferred lubricants are stearates and polyethylene glycol. Additional components that may be incorporated into sublingual and lingual dosage forms are known, or will be apparent, to those skilled in this art (See, e.g., Remington: The Science and Practice of Pharmacy (2000), supra).

Other preferred compositions for sublingual administration include, for example, a bioadhesive to retain a particle composition or delivery vehicle sublingually; a spray, paint, or swab applied to the tongue; or the like. Increased residence time increases the likelihood that the administered invention can be absorbed by the mucosal tissue.

Transdermal administration of a particle composition or delivery vehicle through the skin or mucosal tissue can be accomplished using conventional transdermal drug delivery systems, wherein the agent is contained within a laminated structure (typically referred to as a transdermal “patch”) that serves as a drug delivery device to be affixed to the skin.

Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active agent and any other materials that are present, the backing is preferably made of a sheet or film of a flexible elastomeric material. Examples of polymers that are suitable for the backing layer include polyethylene, polypropylene, polyesters, and the like.

During storage and prior to use, the laminated structure includes a release liner. Immediately prior to use, this layer is removed from the device to expose the basal surface thereof, either the drug reservoir or a separate contact adhesive layer, so that the system may be affixed to the skin. The release liner should be made from a drug/vehicle impermeable material.

Transdermal drug delivery systems may in addition contain a skin permeation enhancer. That is, because the inherent permeability of the skin to some drugs may be too low to allow therapeutic levels of the drug to pass through a reasonably sized area of unbroken skin, it is necessary to coadminister a skin permeation enhancer with such drugs. Suitable enhancers are well known in the art and include, for example, those enhancers listed below in transmucosal compositions.

Formulations can comprise one or more anesthetics. Patient discomfort or phlebitis and the like can be managed using anesthetic at the site of injection. If used, the anesthetic can be administered separately or as a component of the composition. One or more anesthetics, if present in the composition, is selected from the group consisting of lignocaine, bupivacaine, dibucaine, procaine, chloroprocaine, prilocaine, mepivacaine, etidocaine, tetracaine, lidocaine and xylocaine, and salts, derivatives or mixtures thereof.

The present subject matter is further described herein by the following non-limiting examples which further illustrate the invention, and are not intended, nor should they be interpreted to, limit the scope of the invention.

### Examples

### 1. Materials and Methods

a. PRINT Particle Fabrication

Master templates of all the shapes tested were prepared using photolithography. Fluorocur molds were generated from silicon master templates of the particles (Liquidia Technologies). PRINT particles were composed of 96 wt % poly(ethylene glycol) diacrylate (MW: 700), 2 wt % fluorescein-O-acrylate, and 2 wt % 2,2,-diethoxyacetophenone. This polymer mixture was spread onto a mold and a polyvinylpyrrolidone (PVP)-treated poly(ethylene terephthalate) (PET) sheet was laminated on top of the mold and polymer mixture to fill the molded wells. The cover sheet was peeled away from the mold at the nip of the laminator leaving a mold with filled wells of the polymer. The mold was then placed in a UV curing chamber, purged with nitrogen, and cured (λ=365 nm, power >20 mW/cm²) for 4 min. Particles were transferred out of the mold onto a polyvinyl alcohol (PVOH)-treated PET sheet by laminating the mold and PVOH sheet together and running them through a heated laminator. The mold was then peeled off leaving free particles on the PVOH harvest layer. Particles were collected from the harvest sheet by bead harvesting with water. After washing the particles 5 times with water, the particles were pelleted by centrifugation, the supernatant removed, and the pellet was resuspended in tert-butanol and flash frozen with liquid nitrogen. The particles were lyophilized overnight to generate a dry powder.

b. Zeta Potential Measurements

The zeta potential of PRINT particles was measured using a nano ZS zetasizer (Malvern Instruments) in 1 mM KCl.

c. Aerodynamic Diameter Measurement

The mass median aerodynamic diameter (MMAD) was calculated using gmsh software.

d. Uptake Experiments

MH-S (ATCC) and RAW264.7 (ATCC) cell lines were used for the particle uptake experiments. Cells were plated at 20 k cells/well in a 24-well plate 48 hours before dosing. Particles were re-suspended in water and particle number counted with a hemacytometer. Cells were dosed at a constant particle number (10 particles/cell) in DMEM and were incubated together from 0.5 to 24 hr (37° C., 5% CO₂). After incubation, cells were washed with Dulbecco's Phosphate Buffer Saline (PBS) solution and detached with trypsin (MH-S) or cell-scraped (RAW264.7). The cells were then re-suspended in an 0.2% trypan blue solution in PBS to quench extracellular fluorescence. The samples were analyzed by flow cytometry (CyAn ADP, Dako). 5,000 cells were measured for each sample.

e. Confocal Laser Scanning Microscopy

MH-S cells were plated at 5×10⁵ on cover slips in 6-well dishes and grown for 24 hours before dosing. Cells were treated with particles for 0.5 to 12 hours. Cells were then washed with phosphate-buffered saline, pH 7.4 (PBS) and fixed with 4% Para formaldehyde in PBS for 10 min at room temperature. Cells were permeablized with 0.1% triton-X100 in PBS for 3 min and incubated in phalloidin (Alexa-555) Molecular probes for 1 hr RT in dark. Coverslips were washed three times with PBS and mounted with fluor save reagent (Calbiochem). Samples were then analyzed by confocal microscopy.

Confocal images were acquired using a Zeiss 710 laser scanning confocal imaging system (Olympus) fluorescence microscope fitted with a PlanApo 60× oil objective (Olympus). The final composite images were created using Adobe Photoshop CS (Adobe Systems, San Jose, Calif.).

f. Scanning Electron Microscopy

MH-S cells were seeded at 50 k cells/well on glass slides placed in each well of 12-well plates and allowed to adhere overnight. The cells were dosed at a particle concentration of 50 μg/ml and incubated for timepoints between 0.5 and 14 hours. After incubation, cells were fixed with 4% paraformaldehyde (PFA) at room temperature and dehydrated with washes of increasing ethanol concentration. Cells were then run under critical point drying (CPD) conditions. After drying, cells were coated with gold-palladium for SEM imaging.

### 2. Fabrication of Particles

A collection of non-spherical shapes were fabricated using PRINT technology to create a monodisperse and geometrically precise population of particles. Aerodynamically inspired shapes, such as pollen, were also designed to investigate pulmonary deposition and cell internalization properties. These hydrogel micro-particles were primarily composed of cross-linked poly(ethylene glycol) and have calculated aerodynamic diameters between 1-5 μm, the ideal size for pulmonary deposition. Four particle characteristics are used to describe the shapes examined: (1) the shape diameter (SD) which is the minimum diameter of a circumscribed circle around the particle and is the number that precedes the shape name to differentiate different sizes of the same geometry; (2) the minimum feature size (MFS) which is the diameter of the smallest distinct geometry of the shape; (3) the particle volume; and (4) the aerodynamic diameter (MMAD) of the particle.

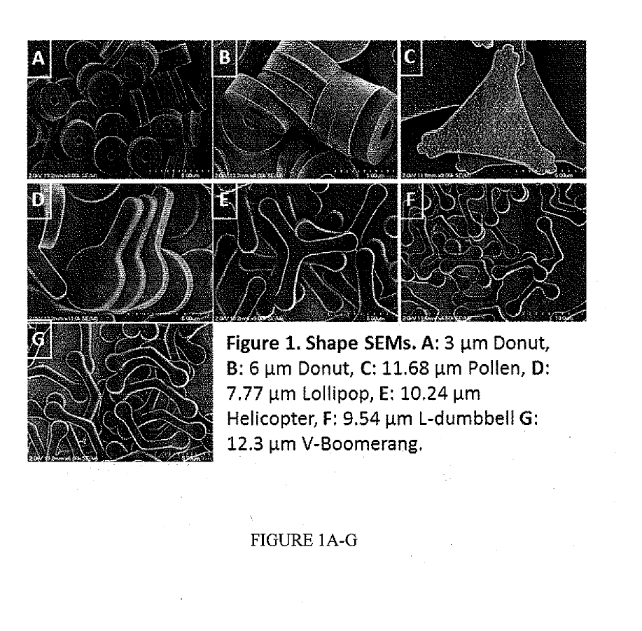

Three distinct families of shapes were examined: (1) a toroid-shaped series (SDs=3 μm, and 6 μm; FIG. 1A,B), (2) a pollen shape (11.68 μm [MFS=1.5 μm]; FIG. 1C), and (3) a ball-and-stick series [7.77 μm lollipop, SD=7.77 μm, MFS=4 μm (FIG. 1D); 10.24 μm helicopter, SD=10.24 μm, MFS=1.6 μm, (FIG. 1E); 9.54 μm l-dumbbell, SD-9.54, MFS=2.5 μm (FIG. 1F); 12.3 μm v-boomerang, SD=12.3 μm, MFS=2.25 μm (FIG. 1G)]. Scanning electron microscopy was used to measure the dimensions of the particles. The measured zeta potential of all particle geometries was around −12 mV.

### 3. Kinetics of Particle Uptake as a Function of Shape and Size

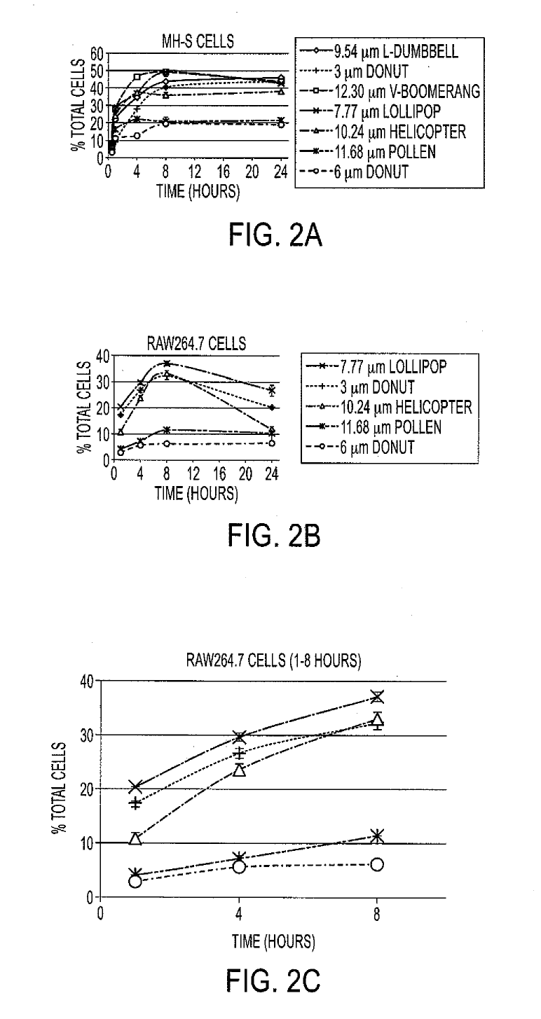

Flow cytometry was used to investigate effects of particle geometry on macrophage internalization. MH-S murine alveolar macrophages were dosed with a constant particle number (10 particles/cell) and uptake was measured from 0.5 to 24 hours at four time points. Kinetics of cellular internalization was analyzed using a flow cytometry method in which membrane-bound and internalized particles were differentiated using fluorescence quenching with trypan blue (19). For particle shapes that were internalized efficiently, a rapid increase in particle-positive cells was observed within the first eight hours after which intracellular particle concentration remained constant (FIG. 2A). The internalization percentage of particles that were less efficiently internalized also leveled out after eight hours, but the initial internalization kinetics were significantly slower. The same trends were also seen when RAW264.7 murine leukaemic macrophages were dosed similarly (FIG. 2B) indicating that the learnings from this work may potentially be utilized in applications targeting macrophages in other parts of the body. The doubling time of the RAW264.7 cells is 12-16 hours, twice as fast as MH-S cells, which accounts for the decrease in the overall percentage of cells with internalized particles at the longer time points.

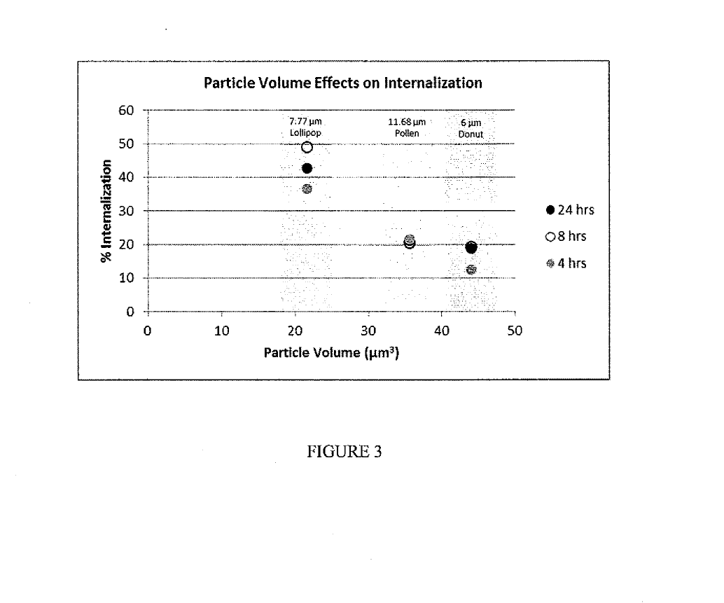

Large porous particles were able to avoid macrophage uptake in the deep lung and increase systemic bioavailability of delivered drugs (11) when compared to smaller, denser particles of the same MMAD. As disclosed herein, particles were generated by rationally designed geometric shapes that encompassed certain characteristics to increase or decrease macrophage phagocytosis. It was observed that different shapes with aerodynamic diameters of 3±0.7 μm could be tailored to have over two-fold difference in macrophage internalization (FIG. 3). Phagocytosis of these PRINT particles was predominantly influenced by volume as shapes of similar volume had similar internalization patterns. After 24 hours, MH-S cells with internalized particles of all of the ball-and-stick series of shapes with volumes of approximately 20 μm³ was approximately 45% while the internalization of 6 μm donut and 11.68 μm pollen geometries, which have volumes greater than 35 μm³, leveled out at 20%. This significant reduction in macrophage uptake is promising for pulmonary therapies targeting cystic fibrosis or asthma which would benefit from enhanced residence time and minimal drug clearance from the lung. These results demonstrate the influence of particle geometry on macrophage uptake and the ability of PRINT to generate particles of similar aerodynamic diameter but vastly different geometric dimensions.

### 4. Particle Orientation and Geometric Effects in Macrophage Phagocytosis

The human lungs constantly survey a numerous and diverse population of particulates. Research into how the cell acts on these particles has been limited by the lack of fabrication methods for generating non-spherical shapes. Budding yeast has been extensively used to investigate the proteins and cellular mechanisms involved in phagocytosis. Champion, et al. stretched polystyrene particles to understand how local contact angle may influence the initiation of phagocytosis (20). Utilizing the unique shapes of PRINT particles, we have determined how features such as asymmetry, appendages, angles, fenestrations, and size affect particle internalization by macrophages.

Macrophage uptake of these particles was examined using a murine alveolar macrophage (MH-S) cell line and RAW264.7 murine leukaemic macrophages. Kinetics of cellular internalization was measured using a flow cytometry method in which membrane-bound and internalized particles were differentiated using fluorescence quenching with trypan blue (17). These data suggests that shape has a significant influence on particle phagocytosis. Differences in internalization were generally segregated by particle diameter (FIG. 2A). However, certain geometries have enhanced uptake profiles, especially particles that have portions or appendage dimensions of approximately 1-3 microns, a length scale associated with most of the commonly known bacterial pathogens.

Particles with maximal diameters of 2 μm had the highest percentage of particles internalized from initial particle number dosed (12). Thus it is interesting to note that all of the shapes with distinct minimum feature sizes equal to or smaller than 3 μm, regardless of total volume, showed similar macrophage uptake from the flow cytometry data. The ball-and-stick series of shapes included geometries with variable number of arms, angle lengths, and end terminal diameter in order to investigate how certain particle characteristics may influence macrophage uptake. While all of the ball-and-stick series of shapes essentially leveled out at 45% internalization at 24 hours there were some uptake differences at earlier time points. Most notably is the faster internalization rate of the 12.3 μm v-boomerangs. This shape most closely mimics a bacterial pathogen with its rod-like shape and does not have a feature which may hinder phagocytosis such as the extended angled arms of the 10.24 μm helicopter or the 4 μm diameter end terminal geometry of the 7.77 μm lollipop.

Of the shapes with volumes greater than 20 μm³ and terminal circular geometries (6 μm donut, helicopter, v-boomerang, lollipop), the amount of internalization increased as the ball diameter decreased (FIG. 2B). Particles with “handles” or arms (such as the v-Boomerang and helicopter shapes) are more rapidly internalized than particles without handles (such as the 6 μm donuts). Scanning electron microscopy imaging provides a 3D analysis of cell-particle associations. Micrographs show that particle geometry indeed matters for cellular uptake. Of the shapes with volumes greater than 20 μm³, the cell shows prominent membrane progression along a terminal end or vertex working its way around the particle while with 6 μm donuts, which do not have arms or vertices, membrane progression around the particle is slow and exhibits more spreading like qualities rather than phagocytosis initiation (FIG. 3). Points of attachment to the particle affect phagocytosis as seen when comparing the 11.68 μm pollen (volume: 50.86 μm³) and 6 μm donuts (volume: 25.87 μm3). Although the pollen is twice the volume and has a larger shape diameter, it is internalized at a similar rate. This may be due to the cell's inability to discriminate total particle size and volume until after initiation of phagocytosis.

The MFS of the 11.68 μm pollen are 1.5 μm which allows the cell to initiate phagocytosis at the vertices. The plateau of phagocytosis kinetics after the cell encounters a feature size greater than 4 μm may explain the similar internalization trends of the 6 μm donuts, 11.68 μm pollen, and 7.77 μm lollipops.

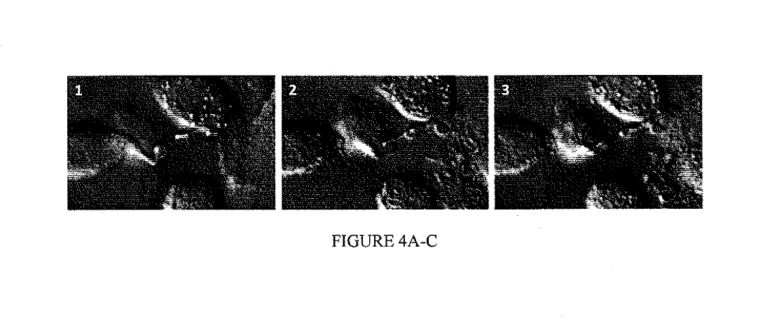

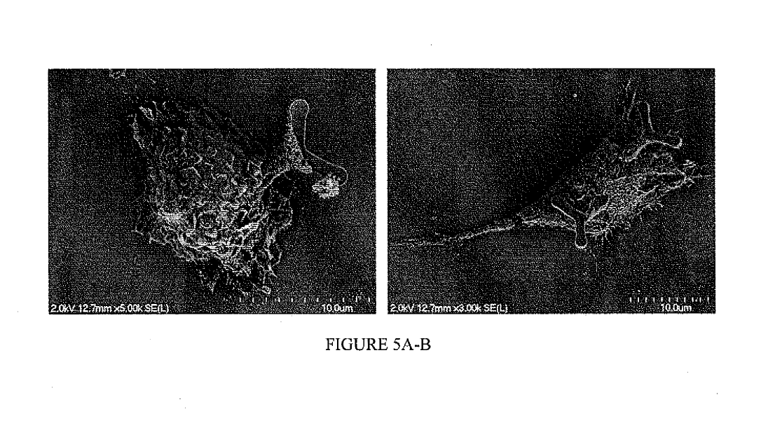

To further investigate these observations, the effect of angled geometries on cell interactions with the particle was visualized using live cell imaging. Internalization kinetics of a particle can decrease when phagocytosis is initiated at the angle versus at the terminal end of an appendage. Live video microscopy of 9.54 μm l-dumbbells interactions with macrophages indeed demonstrates that there is a difference in uptake kinetics dependent on where the cell first contacts the particle (FIG. 4). The particle first contacted by the cell on “ball” section (labeled A) is fully internalized while the particle contacted at the angled “stick” section (labeled B) is still mostly external to the cell membrane. Phagocytosis of curved budding yeast has been studied with Dictyostelium cells in which it was found that the cells scan for concave or convex regions and can switch between actin polymerization and depolymerization to fully engulf, release, or cleave the particle (21). When this observation is integrated with the flow cytometry data, it may be inferred that the 10.24 μm helicopters had slightly lower internalization than the other ball-and-stick shapes due to the 120° angle of the arms. Most of the helicopters had phagocytosis initialized at the terminal end of one of the arms thus as the cell membrane approaches the “Y” of the shape it encounters a wide angle which requires more actin polymerization to fully engulf the particle (FIG. 5).

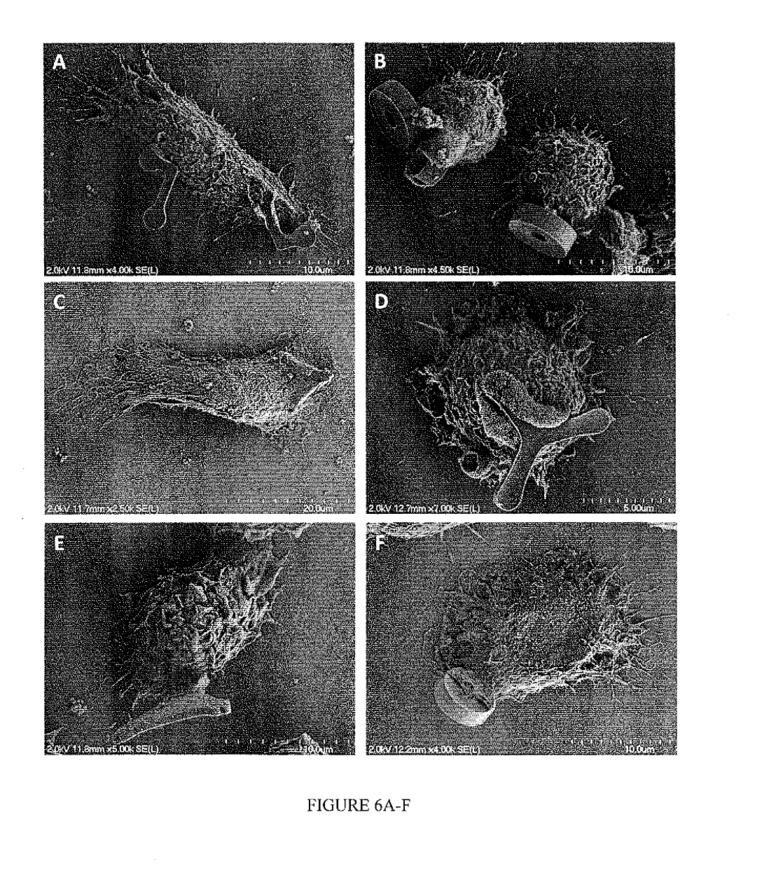

To more clearly examine geometric influences on macrophage uptake, fluorescent microscopy and scanning electron microscopy (SEM) techniques were utilized to capture cell-particle interactions at early time points (FIG. 6). The local particle shape at the point of attachment is observed to influence the kinetics of particle uptake (15). Phagocytosis is an actin-dependent, and thus an energy-dependent, process. The more actin remodeling that the cell must undergo in order to engulf a particle will slow the rate of internalization. This can be seen in the SEM images with the 6 μm donuts and 10.24 μm helicopter shapes which were fixed after a one hour incubation with the particles (FIG. 6A, B). When one cell is associated with two or more particles, the particle in which the local particle contact angle (Ω) is more favorable towards phagocytosis is observed to be further engulfed. However, these micrographs are of fixed cells thus the order and timeline of cell attachment to the particle cannot be definitively determined.

Interestingly, we observed active phagocytosis of these PEG particles when Ω was larger than 45° (FIG. 6C, D). Previous studies have shown that both IgG-opsonized and nonopsonized particles with Ω>45° induced spreading of the cell membrane but did not induce phagocytosis (15). The two most studied pathways of macrophage particle internalization are FcR- and complement-mediated phagocytosis, however, since these particles were nonopsonized the exact mechanism in which they are phagocytosed is not defined.

Complement-mediated phagocytosis involves particles “sinking” into the membrane (22, 23) while FcR-mediated phagocytosis, on the other hand, extends the phagocytic cup around the particle before drawing the engulfed particle into the body of the cell (24, 25). Since complement-mediated phagocytosis involves allowing the particle to sink into the membrane rather than totally enclosing it with actin filaments the strict dependence on local contact angle as a determinant for internalization should be alleviated. It was observed that a majority of particles associated with cells were raised above the plane of the surface as the cell drew it into the membrane (FIG. 6E, F). This may allow the cell to “wrap” around the particle and initiate phagocytosis rather than just spreading along the flat side of the particle. To note, however, most observed particles in the process of being internalized did have Ω<45° which is more favorable for phagocytosis.

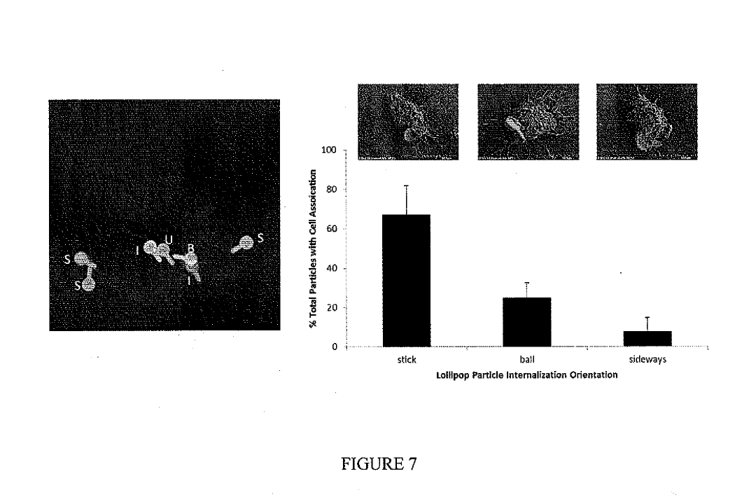

The 7.77 μm lollipop particles are an interesting geometry to investigate due to their asymmetry. With the “ball” end having a diameter of 4 μm and the “stick” end having a diameter of 1 μm, this shape offered a unique test of how particle geometry may dictate the cellular mechanisms of phagocytosis. From the flow cytometry data, the lollipops had similar internalization kinetics as the rest of the ball-and-stick series indicating that having one end significantly larger than another may not affect overall kinetics in macrophage internalization. However, from fluorescent microscopy analysis it was observed that at early time points the macrophages had a preference for the narrow end of the lollipop particles during initiation of particle internalization (FIG. 7). Because the cells are washed with PBS before fixation, unattached or loosely held particles are cleared away. This data may imply that the cell may have a better “grip” on the 1 μm stick end of the lollipops because it initiates phagocytosis (actin remodeling) faster than if it first took hold of the 4 μm “ball” end which requires more time for actin polymerization around the particle although it is not known whether the cell will manipulate the particle as it is drawing it into the cell membrane. This data will be useful in designing particles that de-target macrophages by incorporating larger feature sizes.

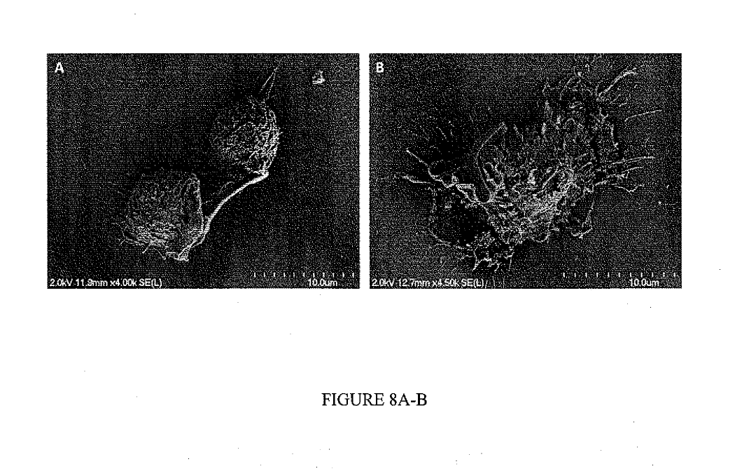

Although the modulus of these highly cross-linked particles was not measured, it is interesting to note that the cells exerted enough force to visibly stretch and bend the particles (FIG. 8). More rigid particles may be preferentially phagocytosed over softer particles (26).

To investigate both the macro- and micro-implications of particle shape in targeting or de-targeting macrophage uptake, using PRINT technology, particles with similar aerodynamic diameters but very different geometries were fabricated. From the flow cytometry data we show that macrophage internalization can be reduced by increasing the volume of the particle. Since particles with similar MMAD's deposit in the same area of the lung, being able to tailor particles to avoid macrophage uptake for specific therapies will undoubtedly increase the efficiency of pulmonary drug delivery. In order to more fully understand shape dynamics on macrophage uptake we looked at the influence of individual geometric features. Previous studies on shape have shown that local particle contact angle influences the initiation of phagocytosis. We found that while internalization kinetics were correlated to Ω, local contact angles greater than 45° did not necessarily prevent the initiation of phagocytosis. The inclusion of angles or asymmetry in the particles was shown to have an influence on macrophage uptake.

These studies collectively demonstrate the influence of particle geometry on macrophage phagocytosis. Particle volume may have less influence on phagocytosis initiation than local particle shape at the macrophage point of contact. Particles with terminal end geometries less than 3 μm were internalized more quickly than their volume or overall shape diameter might indicate. Thus, in order to target macrophages, geometries with multiple arms and end terminal geometries less than 3 μm would be preferred. To de-target macrophages, preferred particles would have feature sizes greater than 4 μm. Appendages may be present or absent. One preferred particle has feature sizes greater than 4 μm but does not contain an appendage. As the aerodynamic diameters of all particles tested are in the range for pulmonary deposition (1-5 μm), these results emphasize the ability of particle geometry to confer additional efficacy to inhaled drug delivery vehicles by targeting and/or de-targeting alveolar macrophages.

Because the PRINT fabrication method allows for particle compositions of pure drug and other biodegradable components, different therapeutics can be incorporated into these geometries and efficacy based on shape can be further investigated. Preferably, the subject matter disclosed herein is applied to pulmonary delivery applications resulting in more efficient drug carriers to the lung. Particles fabricated using the PRINT method have very specific geometries that can be tailored to target and de-target macrophages. This will impart the ability to increase the efficacy of delivered drugs in particular through the pulmonary route. This can reduce side effects related to pulmonary drug delivery such as unintended absorption of the drug into the systemic circulation by delivering the drug in a geometric shape that encourages macrophage uptake. The invention may also be used to increase drug bioavailability by engaging macrophages with a “dummy” particle such that the target therapeutic is able to circumvent phagocytosis and can be absorbed into the systemic circulation. Adding a macrophage targeting component to these aerodynamic drug particles may impart unprecedented control over deposition and efficacy of therapeutics in the lung.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### BIBLIOGRAPHY

1. Chow A H, Tong H H, Chattopadhyay P, & Shekunov B Y (2007) Particle engineering for pulmonary drug delivery. Pharm Res 24(3):411-437.
2. Rolland J P, et al. (2005) Direct Fabrication and Harvesting of Monodisperse, Shape-Specific Nanobiomaterials. Journal of the American Chemical Society 127(28):10096-10100.
3. Euliss L E, DuPont J A, Gratton S, & DeSimone J (2006) Imparting size, shape, and composition control of materials for nanomedicine. Chemical Society Reviews 35(11):1095-1095.
4. Chan H K, Clark A, Gonda I, Mumenthaler M, & Hsu C (1997) Spray dried powders and powder blends of recombinant human deoxyribonuclease (rhDNase) for aerosol delivery. Pharm Res 14(4):431-437.
5. Oberdörster G (1988) Lung Clearance of Inhaled Insoluble and Soluble Particles. Journal of Aerosol Medicine 1(4):289-330.
6. Edsbacker S, et al. (2008) Do airway clearance mechanisms influence the local and systemic effects of inhaled corticosteroids? Pulm Pharmacol Ther 21(2):247-258.
7. Suarez S & Hickey A J (2000) Drug properties affecting aerosol behavior. Respir Care 45(6):652-666.
8. Lombry C, Edwards Da, Préat V, & Vanbever R (2004) Alveolar macrophages are a primary barrier to pulmonary absorption of macromolecules. American journal of physiology. Lung cellular and molecular physiology 286(5):L1002-1008.
9. Sharma R, Saxena D, Dwivedi A K, & Misra A (2001) Inhalable microparticles containing drug combinations to target alveolar macrophages for treatment of pulmonary tuberculosis. Pharm Res 18(10):1405-1410.
10. Leemans J C, et al. (2001) Depletion of alveolar macrophages exerts protective effects in pulmonary tuberculosis in mice. Journal of immunology (Baltimore, Md.: 1950) 166(7):4604-4611.
11. Edwards D A, et al. (1997) Large porous particles for pulmonary drug delivery. Science 276(5320):1868-1871.
12. Tabata Y & Ikada Y (1988) Effect of the size and surface charge of polymer microspheres on their phagocytosis by macrophage. Biomaterials 9(4):356-362.
13. El-Sherbiny I M, McGill S, & Smyth H D (2010) Swellable microparticles as carriers for sustained pulmonary drug delivery. J Pharm Sci 99(5):2343-2356.
14. Geng Y A N, et al. (2007) Shape effects of filaments versus spherical particles in flow and drug delivery. Nature nanotechnology 2(4):249-255.
15. Champion J A & Mitragotri S (2006) Role of target geometry in phagocytosis. Proceedings of the National Academy of Sciences of the United States of America 103(13):4930-4934.
16. Sharma G, et al. (2010) Polymer particle shape independently influences binding and internalization by macrophages. Journal of Controlled Release 147(3):408-412.
17. Enlow E M, Luft J C, Napier M E, & DeSimone J M (2011) Potent engineered PLGA nanoparticles by virtue of exceptionally high chemotherapeutic loadings. Nano Lett 11(2):808-813.
18. Merkel T J, et al. (2011) Using mechanobiological mimicry of red blood cells to extend circulation times of hydrogel microparticles. Proc Natl Acad Sci USA 108(2):586-591.
19. Fattorossi A, Nisini R, Pizzolo J G, & D'Amelio R (1989) New, simple flow cytometry technique to discriminate between internalized and membrane-bound particles in phagocytosis. Cytometry 10(3):320-325.
20. Champion J A & Mitragotri S (2009) Shape Induced Inhibition of Phagocytosis of Polymer Particles. Pharmaceutical research 26(1):244-249.
21. Clarke M, et al. (2010) Curvature recognition and force generation in phagocytosis. BMC Biol 8:154.
22. May R C & Machesky L M (2001) Phagocytosis and the actin cytoskeleton. J Cell Sci 114(Pt 6):1061-1077.
23. Allen L A & Aderem A (1996) Molecular definition of distinct cytoskeletal structures involved in complement- and Fc receptor-mediated phagocytosis in macrophages. J Exp Med 184(2):627-637.
24. Aderem A & Underhill D M (1999) Mechanisms of Phagocytosis in Macrophages. Annual Review of Immunology 17(1):593-623.
25. Swanson J A (2008) Shaping cups into phagosomes and macropinosomes. Nat Rev Mol Cell Biol 9(8):639-649.
26. Beningo K A & Wang Y L (2002) Fc-receptor-mediated phagocytosis is regulated by mechanical properties of the target. J Cell Sci 115(Pt 4):849-856.

1. A drug delivery device that exhibits reduced uptake by macrophages, comprising:
a particle having an engineered geometry comprising a body member and an appendage protruding from said body member, wherein said engineered geometry is configured and dimensioned to hinder phagocytosis by a macrophage.

2. (canceled)

3. (canceled)

4. The drug delivery device of claim 1, wherein said appendage comprises a width to length ratio of greater than 1:2.

5. The drug delivery device of claim 1, wherein said appendage has a width to length ratio of greater than 1:4.

6. The drug delivery device of claim 1, wherein said appendage protrudes at least about 4 micrometers in length from said body member.

7. The drug delivery device of claim 1, further comprising a second appendage protruding from said body member.

8. The drug delivery device of claim 1, wherein the particle comprises a polymer, a monomer, a plurality of monomers, a polymerization initiator, a polymerization catalyst, an inorganic precursor, a pharmaceutical agent, a tag, a magnetic material, a paramagnetic material, a superparamagnetic material, a ligand, a cell penetrating peptide, a porogen, a surfactant, a charged species, or a biologic.

9. The drug delivery device of claim 1, wherein said engineered geometry is substantially a toroid-shape, substantially a ball-and-stick shape, substantially a helicopter shape, substantially a pollen-shape, substantially a dumbbell-shape, or substantially a boomerang-shape.

10. The drug delivery device of claim 1, wherein said particle has a ratio of total volume to calculated aerodynamic diameter of at least about 1.

11. The drug delivery device of claim 8, wherein said ratio is between about 1 and about 20.

12. A method of hindering phagocytosis of an agent by a macrophage comprising,
administering a plurality of particles wherein each particle of the plurality has an engineered geometry comprising a body member and an appendage protruding from said body member, said particle further comprising an agent, wherein said particle exhibits reduced uptake by macrophages compared to a substantially spherical particle having substantially the same volume as the engineered particle.

13. The method of claim 12, wherein at least about 50% of said particles have not been phagocytized by a macrophage at about 24 hours after said administration.

14. The method of claim 12, wherein at least about 60% of said particles have not been phagocytized by a macrophage at about 24 hours after said administration.

15. The method of claim 12, wherein said appendage is configured with a width to length ratio of greater than about 1:2, wherein said agent is released from said appendage.

16. The method of claim 15, wherein said macrophage is an alveolar macrophage.

17. A method of selecting internalization kinetics of a particle comprising,
a) assessing internalization kinetics of a particle by a macrophage, wherein said particle has a distinct engineered feature comprising a body member and an appendage protruding from said body member,
b) assessing internalization kinetics of a second particle by a macrophage, wherein said second particle has at least one distinct engineered feature comprising a body member and an appendage protruding from said body member and differing from the engineered feature of the particle in a) in at least one aspect;
c) comparing the internalization kinetics in a) and b); and
d) preparing an engineered particle comprising a body member and an appendage protruding from said body member based on the comparison in c).

18. The method of claim 17, wherein b) further comprises assessing the internalization kinetics of additional particles, wherein each particle has at least one distinct engineered feature comprising a body member and an appendage protruding from said body member, and c) further comprises comparing the internalization kinetics of all particles.

19. (canceled)

20. The drug delivery device of claim 1, wherein said particles are delivered to the lung of a patient.

21. The drug particle of claim 1, wherein said body member has a width greater than twice the width of said appendage.

22. The drug particle of claim 1, wherein said appendage branches into at least two appendages.
